(19) United States
(12) Patent Application Publication
(10) Pub. No.: US 2009/0176839 A1
(43) Pub. Date: Jul. 9, 2009

(54) FORMULATIONS OF 3-(6-(1-(2,2-DIFLUOROBENZO[D][1,3]DIOXOL-5-YL) CYCLOPROPANECARBOXAMIDO)-3-METHYLPYRIDIN-2-YL)BENZOIC ACID

(76) Inventors: Ali Keshavarz-Shokri, San Diego, CA (US); Christopher Young, Waltham, MA (US)

Correspondence Address: VERTEX PHARMACEUTICALS INC., 130 WAVERLY STREET, CAMBRIDGE, MA, 02139-4242, US

(21) Appl. No.: 12/327,922

(22) Filed: Dec. 4, 2008

Related U.S. Application Data
(60) Provisional application No. 61/012,168, filed on Dec. 7, 2007.

(51) Int. Cl. A61K 31/443; A61P 43/00

(52) U.S. Cl. 514/338

(57) ABSTRACT

The present invention relates to formulations of 3-(6-(1-(2,2-difluorobenzo[d][1,3]dioxol-5-yl)cyclopropanecarboxamido)-3-methylpyridin-2-yl)benzoic acid, pharmaceutical packs or kits thereof, and methods of treatment therewith.

### CROSS REFERENCE TO RELATED APPLICATIONS

This application claims the benefit under 35 U.S.C. § 119 to U.S. provisional patent application Ser. No. 61/012,168, filed Dec. 7, 2007, the entire contents of which are incorporated herein by reference.

### TECHNICAL FIELD OF THE INVENTION

The present invention relates to an oral formulation comprising substantially free 3-(6-(1-(2,2-difluorobenzo[d][1,3]dioxol-5-yl)cyclopropanecarboxamido)-3-methylpyridin-2-yl)benzoic acid (Compound 1) as described herein, water, and a viscosity agent. The oral formulation may additionally comprise a surfactant, antifoaming agent, buffer, and taste masker. The invention further relates to a method of treating a CFTR mediated disease such as cystic fibrosis with such a formulation.

### BACKGROUND OF THE INVENTION

CFTR is a cAMP/ATP-mediated anion channel that is expressed in a variety of cells types, including absorptive and secretory epithelia cells, where it regulates anion flux across the membrane, as well as the activity of other ion channels and proteins. In epithelia cells, normal functioning of CFTR is critical for the maintenance of electrolyte transport throughout the body, including respiratory and digestive tissue. CFTR is composed of approximately 1480 amino acids that encode a protein made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

The gene encoding CFTR has been identified and sequenced (See Gregory, R. J. et al. (1990) Nature 347:382-386; Rich, D. P. et al. (1990) Nature 347:358-362), (Riordan, J. R. et al. (1989) Science 245:1066-1073). A defect in this gene causes mutations in CFTR resulting in cystic fibrosis (“CF”), the most common fatal genetic disease in humans. Cystic fibrosis affects approximately one in every 2,500 infants in the United States. Within the general United States population, up to 10 million people carry a single copy of the defective gene without apparent ill effects. In contrast, individuals with two copies of the CF associated gene suffer from the debilitating and fatal effects of CF, including chronic lung disease.

In patients with cystic fibrosis, mutations in CFTR endogenously expressed in respiratory epithelia leads to reduced apical anion secretion causing an imbalance in ion and fluid transport. The resulting decrease in anion transport contributes to enhanced mucus accumulation in the lung and the accompanying microbial infections that ultimately cause death in CF patients. In addition to respiratory disease, CF patients typically suffer from gastrointestinal problems and pancreatic insufficiency that, if left untreated, results in death. In addition, the majority of males with cystic fibrosis are infertile and fertility is decreased among females with cystic fibrosis. In contrast to the severe effects of two copies of the CF associated gene, individuals with a single copy of the CF associated gene exhibit increased resistance to cholera and to dehydration resulting from diarrhea—perhaps explaining the relatively high frequency of the CF gene within the population.

Sequence analysis of the CFTR gene of CF chromosomes has revealed a variety of disease causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S et al. (1990) Proc. Natl. Acad. Sci. USA 87:8447-8451). To date, >1000 disease causing mutations in the CF gene have been identified (http://www.genet.sickkids.on.ca/cftr/). The most prevalent mutation is a deletion of phenylalanine at position 508 of the CFTR amino acid sequence, and is commonly referred to as ΔF508-CFTR. This mutation occurs in approximately 70% of the cases of cystic fibrosis and is associated with a severe disease.

The deletion of residue 508 in ΔF508-CFTR prevents the nascent protein from folding correctly. This results in the inability of the mutant protein to exit the ER, and traffic to the plasma membrane. As a result, the number of channels present in the membrane is far less than observed in cells expressing wild-type CFTR. In addition to impaired trafficking, the mutation results in defective channel gating. Together, the reduced number of channels in the membrane and the defective gating lead to reduced anion transport across epithelia leading to defective ion and fluid transport. (Quinton, P. M. (1990), FASEB J. 4: 2709-2727). Studies have shown, however, that the reduced numbers of ΔF508-CFTR in the membrane are functional, albeit less than wild-type CFTR. (Dalemans et al. (1991), Nature Lond. 354: 526-528; Denning et al., supra; Pasyk and Foskett (1995), J. Cell. Biochem. 270: 12347-50). In addition to ΔF508-CFTR, other disease causing mutations in CFTR that result in defective trafficking, synthesis, and/or channel gating could be up- or down-regulated to alter anion secretion and modify disease progression and/or severity.

Although CFTR transports a variety of molecules in addition to anions, it is clear that this role (the transport of anions) represents one element in an important mechanism of transporting ions and water across the epithelium. The other elements include the epithelial Na⁺ channel, ENaC, Na⁺/2Cl⁻/K⁺ co-transporter, Na⁺—K⁺-ATPase pump and the basolateral membrane K⁺ channels, that are responsible for the uptake of chloride into the cell.

These elements work together to achieve directional transport across the epithelium via their selective expression and localization within the cell. Chloride absorption takes place by the coordinated activity of ENaC and CFTR present on the apical membrane and the Na⁺—K⁺-ATPase pump and Cl⁻ channels expressed on the basolateral surface of the cell. Secondary active transport of chloride from the luminal side leads to the accumulation of intracellular chloride, which can then passively leave the cell via Cl⁻ channels, resulting in a vectorial transport. Arrangement of Na⁺/2Cl⁻/K⁺ co-transporter, Na⁺—K⁺-ATPase pump and the basolateral membrane K⁺ channels on the basolateral surface and CFTR on the luminal side coordinate the secretion of chloride via CFTR on the luminal side. Because water is probably never actively transported itself, its flow across epithelia depends on tiny transepithelial osmotic gradients generated by the bulk flow of sodium and chloride.

As discussed above, it is believed that the deletion of residue 508 in ΔF508-CFTR prevents the nascent protein from folding correctly, resulting in the inability of this mutant protein to exit the ER, and traffic to the plasma membrane. As a result, insufficient amounts of the mature protein are present at the plasma membrane and chloride transport within epithelial tissues is significantly reduced. Infact, this cellular phenomenon of defective ER processing of ABC transporters by the ER machinery, has been shown to be the underlying basis not only for CF disease, but for a wide range of other isolated and inherited diseases. The two ways that the ER machinery can malfunction is either by loss of coupling to ER export of the proteins leading to degradation, or by the ER accumulation of these defective/misfolded proteins [Aridor M, et al., Nature Med., 5(7), pp 745-751 (1999); Shastry, B. S., et al., Neurochem. International, 43, pp 1-7 (2003); Rutishauser, J., et al., Swiss Med Wkly, 132, pp 211-222 (2002); Morello, J P et al., TIPS, 21, pp. 466-469 (2000); Bross P., et al., Human Mut., 14, pp. 186-198 (1999)].

3-(6-(1-(2,2-Difluorobenzo[d][1,3]dioxol-5-yl)cyclopropanecarboxamido)-3-methylpyridin-2-yl)benzoic acid in salt form is disclosed in International PCT Publication WO 2007056341 (said publication being incorporated herein by reference in its entirety) as a modulator of CFTR activity and thus useful in treating CFTR-mediated diseases such as cystic fibrosis. However, there is a need for stable forms of modulators of CFTR activity, such as Compound 1, that can be used to modulate the activity of CFTR in the cell membrane of a mammal. For ease of use and patient comfort, there is also a need for stable, oral formulations of Compound 1 that can be used to administer effective doses of Compound 1 to the patient.

### SUMMARY OF THE INVENTION

The present invention relates to oral formulations of 3-(6-(1-(2,2-difluorobenzo[d][1,3]dioxol-5-yl)cyclopropanecarboxamido)-3-methylpyridin-2-yl)benzoic acid which has the structure below:

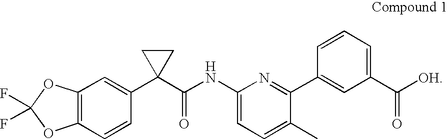

Compound 1 is useful for treating or lessening the severity of a variety of CFTR mediated diseases. Compound 1 may exist in a substantially crystalline and salt free form referred to as Form I as described and characterized herein.

Difficult to wet pharmaceutically acceptable compounds can be problematic in the pharmaceutical arts from a formulations perspective. For example, Compound 1, in addition to having low solubility, is difficult to wet with an aqueous medium, and thereby presents special problems for forming an aqueous dispersion.

Owing to difficulties in wetting Compound 1, the material is difficult to adequately suspend in an aqueous medium without having to resort to using long periods of high shear mixing. One approach to improving the anti-settling properties of a suspension is to use a viscosity agent such as any of the natural gums or cellulosics, such as methylcellulose, to increase viscosity, and thereby retard the rate of re-settling of wetted particles in the suspension. For stability and ease of processing, it may also be desirable to include other agents such as a surfactant, an antifoaming agent, and buffer. For patient comfort it is also desirable to include a taste masker to hide an unpleasant taste associated with Compound 1.

Thus, a good suspension of Compound 1 which maintains an improved shelf life (i.e., which maintains a longer period of suspension prior to re-settling) would represent a valuable addition to the formulations arts. A suspension with improved taste would be a further valuable addition. By “good suspension” it is meant (1) that in an oral formulation according to the invention there is no visible settling for greater than 24 hours at room temperature (RT, usually 25° C.), preferably for greater than one week and (2) that when visible settling does occur, resuspension is easily effected by simple physical mixing such as gentle manual stirring or moderate manual shaking, high shear mixing not being required.

### BRIEF DESCRIPTION OF THE DRAWINGS

FIG. 1 is an X-ray diffraction pattern calculated from a single crystal structure of Compound 1 in Form I.

FIG. 2 is an actual X-ray powder diffraction pattern of Compound 1 in Form I.

FIG. 3 is an overlay of an X-ray diffraction pattern calculated from a single crystal of Compound 1 in Form I, and an actual X-ray powder diffraction pattern of Compound 1 in Form I.

FIG. 4 is a differential scanning calorimetry (DSC) trace of Compound 1 in Form I.

FIG. 5 is a conformational picture of Compound 1 in Form I based on single crystal X-ray analysis.

FIG. 6 is a conformational picture of Compound 1 in Form I based on single crystal X-ray analysis as a dimer formed through the carboxylic acid groups.

FIG. 7 is a conformational picture of Compound 1 in Form I based on single crystal X-ray analysis showing that the molecules are stacked upon each other.

FIG. 8 is conformational picture of Compound 1 in Form I based on single crystal X-ray analysis showing a different view (down a).

FIG. 9 is an overlay of X-ray powder diffraction patterns of 3-(6-(1-(2,2-difluorobenzo[d][1,3]dioxol-5-yl)cyclopropanecarboxamido)-3-methylpyridin-2-yl)benzoic acid.HCl and the same compound after being suspended in an aqueous methylcellulose formulation for 24 hours at room temperature.

FIG. 10 is an overlay of DSC of 3-(6-(1-(2,2-difluorobenzo[d][1,3]dioxol-5-yl)cyclopropanecarboxamido)-3-methylpyridin-2-yl)benzoic acid.HCl and the same compound after being suspended in an aqueous methylcellulose-polysorbate 80 formulation for 0 and 24 hours at room temperature.

FIG. 11 is an ¹HNMR analysis of Compound 1 suspension at T(0).

FIG. 12 is an ¹HNMR analysis of Compound 1 suspension stored at room temperature for 24 hours.

FIG. 13 is an ¹HNMR analysis of 3-(6-(1-(2,2-difluorobenzo[d][1,3]dioxol-5-yl)cyclopropanecarboxamido)-3-methylpyridin-2-yl)benzoic acid.HCl standard.

FIG. 14 is a graph of tissue distribution of Compound 1 in Form I in male rats at 1 to 48 hours following single oral administration at a dose of 75 mg/kg.

### DETAILED DESCRIPTION OF THE INVENTION

### Definitions

As used herein, the following definitions shall apply unless otherwise indicated.

The term “CFTR” as used herein means cystic fibrosis transmembrane conductance regulator or a mutation thereof capable of regulator activity, including, but not limited to, AF508 CFTR and G551D CFTR (see, e.g., http://www.genet.sickkids.on.ca/cftr/, for CFTR mutations).

As used herein “crystalline” refers to compounds or compositions where the structural units are arranged in fixed geometric patterns or lattices, so that crystalline solids have rigid long range order. The structural units that constitute the crystal structure can be atoms, molecules, or ions. Crystalline solids show definite melting points.

As used herein, a “dispersion” refers to a disperse system in which one substance, the dispersed phase, is distributed, in discrete units, throughout a second substance (the continuous phase or vehicle). The size of the dispersed phase can vary considerably (e.g. colloidal particles of nanometer dimension, to multiple microns in size). In one embodiment, the aqueous formulations of the present invention are a dispersion of Compound 1 in water.

The term “modulating” as used herein means increasing or decreasing, e.g. activity, by a measurable amount.

In one aspect, the present invention relates to an aqueous formulation comprising 3-(6-(1-(2,2-difluorobenzo[d][1,3]dioxol-5-yl)cyclopropanecarboxamido)-3-methylpyridin-2-yl)benzoic acid (Compound 1), water, and a viscosity agent.

In another embodiment, Compound 1 is characterized by one or more peaks at 15.2 to 15.6 degrees, 16.1 to 16.5 degrees, and 14.3 to 14.7 degrees in an X-ray powder diffraction obtained using Cu K alpha radiation.

In another embodiment, Compound 1 is characterized by one or more peaks at 15.4, 16.3, and 14.5 degrees.

In another embodiment, Compound 1 is further characterized by a peak at 14.6 to 15.0 degrees.

In another embodiment, Compound 1 is further characterized by a peak at 14.8 degrees.

In another embodiment, Compound 1 is further characterized by a peak at 17.6 to 18.0 degrees.

In another embodiment, Compound 1 is further characterized by a peak at 17.8 degrees.

In another embodiment, Compound 1 is further characterized by a peak at 16.4 to 16.8 degrees.

In another embodiment, Compound 1 is further characterized by a peak at 16.4 to 16.8 degrees.

In another embodiment, Compound 1 is further characterized by a peak at 16.6 degrees.

In another embodiment, Compound 1 is further characterized by a peak at 7.6 to 8.0 degrees.

In another embodiment, Compound 1 is further characterized by a peak at 7.8 degrees.

In another embodiment, Compound 1 is further characterized by a peak at 25.8 to 26.2 degrees.

In another embodiment, Compound 1 is further characterized by a peak at 26.0 degrees.

In another embodiment, Compound 1 is further characterized by a peak at 21.4 to 21.8 degrees.

In another embodiment, Compound 1 is further characterized by a peak at 21.6 degrees.

In another embodiment, Compound 1 is further characterized by a peak at 23.1 to 23.5 degrees.

In another embodiment, Compound 1 is further characterized by a peak at 23.3 degrees.

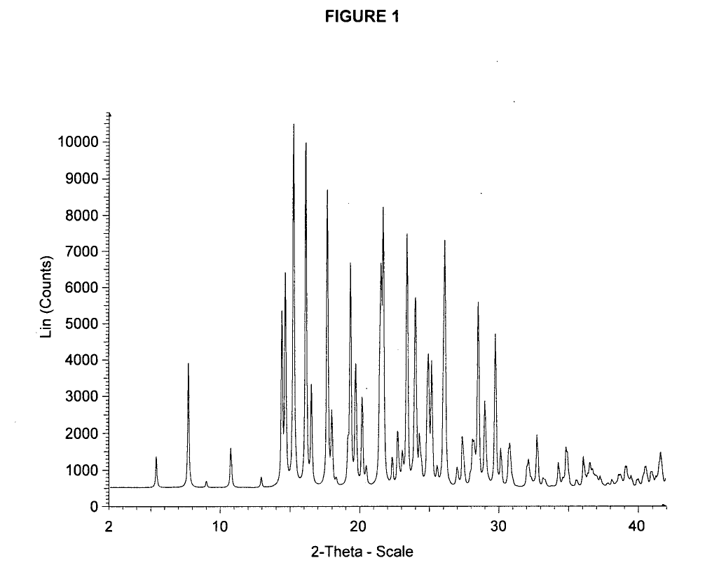

In some embodiments, Compound 1 is characterized by a diffraction pattern substantially similar to that of FIG. 1.

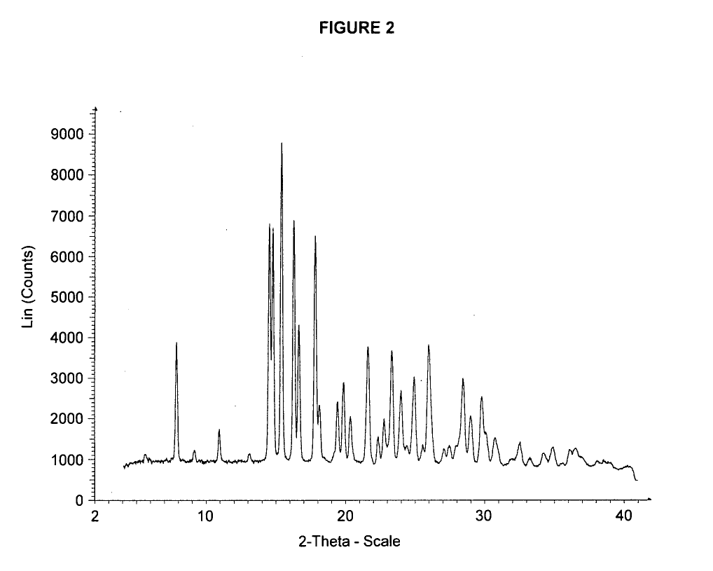

In some embodiments, Compound 1 is characterized by a diffraction pattern substantially similar to that of FIG. 2.

In another embodiment, Compound 1 has a monoclinic crystal system, a P2₁/n space group, and the following unit cell dimensions: a=4.9626 (7) Å; b=12.2994 (18) Å; c=33.075 (4) Å; α=90°; β=93.938 (9)°; and γ=90°.

In another embodiment, the viscosity agent is selected from the group consisting of methyl cellulose, sodium carboxymethylcellulose, hydroxypropylmethyl cellulose, hydroxypropyl cellulose, sodium alginate, polyacrylate, povidone, acacia, guar gum, xanthan gum, tragacanth, and magnesium aluminum silicate. In another embodiment, the viscosity agent is methyl cellulose.

In another embodiment, the concentration of Compound 1 is from about 0.5 to about 20% by weight. In another embodiment, the concentration of Compound 1 is from about 1 to about 10% by weight. In another embodiment, the concentration of Compound 1 is from about 2.5 to about 3.5% by weight.

In another embodiment, the concentration of viscosity agent is from about 0.1 to about 2% by weight. In another embodiment, the concentration of viscosity agent is from about 0.1 to about 1% by weight. In another embodiment, the concentration of viscosity agent is about 0.5% by weight.

In another embodiment, the concentration of Compound 1 is from about 0.5 to about 20% by weight; and the concentration of viscosity agent is from about 0.1 to about 2% by weight. In another embodiment, the concentration of Compound 1 is from about 1 to about 10% by weight; and the concentration of viscosity agent is from about 0.5 to about 1% by weight. In another embodiment, the concentration of Compound 1 is from about 2.5 to about 3.5% by weight; and the concentration of viscosity agent is about 0.5% by weight.

In another embodiment, the concentration of Compound 1 is from about 0.5 to about 20% by weight; and the viscosity agent is methylcellulose at about 0.5% by weight.

In another embodiment, any of the above formulations further comprises a surfactant. In another embodiment, the surfactant is an anionic, cationic, or nonionic surfactant. In another embodiment, the surfactant is an anionic surfactant selected from the group consisting of salts of dodecyl sulfate, lauryl sulfate, laureth sulfate, alkyl benzene sulfonates, butanoic acid, hexanoic acid, octanoic acid, decanoic acid, lauric acid, myristic acid, palmitic acid, stearic acid, arachidic acid, behenic acid, myristoleic acid, palmitoleic acid, oleic acid, linoleic acid, alpha-linolenic acid, arachidonic acid, eicosapentaenoic acid, erucic acid, and docosahexaenoic acid. In another embodiment, the surfactant is a cationic surfactant selected from the group consisting of cetyl trimethylammonium bromide, cetylpyridinium chloride, polethoxylated tallow amine, benzalkonium chloride, and benzethonium chloride. In another embodiment, the surfactant is a nonionic surfactant selected from the group consisting of polysorbate 20, polysorbate 40, polysorbate 60, polysorbate 65, polysorbate 80, alkyl poly(ethylene oxide), poloxamine, alkyl polyglucosides, octyl glucoside, decyl maltoside, fatty alcohol, cetyl alcohol, oleyl alcohol, cocamide MEA, cocamide DEA, and cocamide TEA. In another embodiment, the surfactant is polysorbate 80.

In another embodiment, the concentration of surfactant is from about 0.1 to about 10% by weight. In another embodiment, the concentration of surfactant is from about 0.1 to about 1% by weight. In another embodiment, the concentration of surfactant is about 0.5% by weight. In another embodiment, the surfactant is polysorbate 80 at about 0.5% by weight.

In another embodiment, any of the above formulations further comprises an antifoaming agent. In another embodiment, the antifoaming agent comprises polydimethylsiloxane. In another embodiment, the antifoaming agent is simethicone.

In another embodiment, the concentration of antifoaming agent is from about 0.01 to about 0.2% by weight. In another embodiment, the concentration of antifoaming agent is from about 0.01% to about 0.1% by weight. In another embodiment, the concentration of antifoaming agent is about 0.05% by weight.

In another embodiment, any of the above formulations further comprises a buffer. In another embodiment, the buffer comprises sodium, potassium or ammonium salt of acetic, boric, carbonic, phosphoric, succinic, malic, tartaric, citric, acetic, benzoic, lactic, glyceric, gluconic, glutaric or glutamic acids. In another embodiment, the buffer comprises sodium, potassium or ammonium salt of citric acid.

In another embodiment, any of the above formulations further comprises a masking and/or flavoring agent.

In another aspect, the present invention relates to a method of treating cystic fibrosis in a mammal comprising administering any of the above formulations of Compound 1. In another embodiment, the method comprises administering an additional therapeutic agent. In another embodiment, the additional therapeutic agent is selected from the group consisting of mucolytic agent, bronchodialator, an anti-biotic, an anti-infective agent, an anti-inflammatory agent, a CFTR modulator other than a compound of the present invention, and a nutritional agent.

In another embodiment, the dosage amount of Compound 1 in the dosage unit form is from about 100 mg to about 1,000 mg. In another embodiment, the dosage amount of Compound 1 is from about 200 mg to about 900 mg. In another embodiment, the dosage amount of Compound 1 is from about 300 mg to 8 about 00 mg. In another embodiment, the dosage amount of Compound 1 is from about 400 mg to about 700 mg. In another embodiment, the dosage amount of Compound 1 is from about 500 mg to about 600 mg.

In another aspect, the present invention relates to a pharmaceutical pack or kit comprising any of the above formulations of Compound 1 and instructions for use thereof.

In another aspect, the present invention relates to an oral formulation comprising Compound 1, water, methyl cellulose, polysorbate 80, and simethicone.

In another embodiment, Compound 1 is present in a concentration of about 2.5% to about 3.5% by weight. In another embodiment, the methyl cellulose is present in a concentration of about 0.5% by weight. In another embodiment, the polysorbate 80 is present in a concentration of about 0.5% by weight. In another embodiment, the simethicone is present in a concentration of about 0.05% by weight.

Processes described herein can be used to prepare the compositions of this invention. The amounts and the features of the components used in the processes would be as described herein.

Methods of Preparing Compound 1.

Compound 1 is 3-(6-(1-(2,2-difluorobenzo[d][1,3]dioxol-5-yl)cyclopropanecarboxamido)-3-methylpyridin-2-yl)benzoic acid and in one embodiment can be prepared by coupling an acid chloride moiety with an amine moiety according to Schemes 1-3. Compound 1 in Form I, in one embodiment, is prepared from dispersing or dissolving a salt form, such as HCl, of 3-(6-(1-(2,2-difluorobenzo[d][1,3]dioxol-5-yl)cyclopropanecarboxamido)-3-methylpyridin-2-yl)benzoic acid in an appropriate solvent for an effective amount of time. In another embodiment, Compound 1 in Form I is formed directly from 3-(6-(1-(2,2-difluorobenzo[d][1,3]dioxol-5-yl)cyclopropanecarboxamido)-3-methylpyridin-2-yl)-t-butylbenzoate and an appropriate acid, such as formic acid.

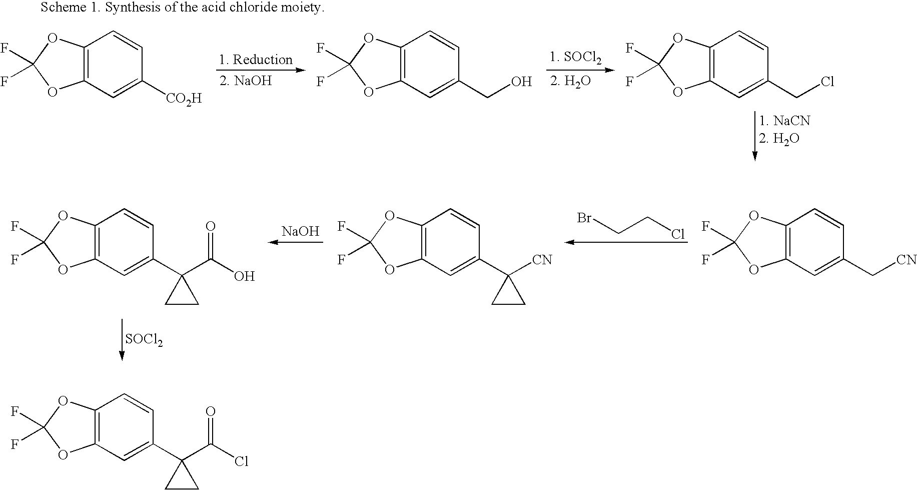

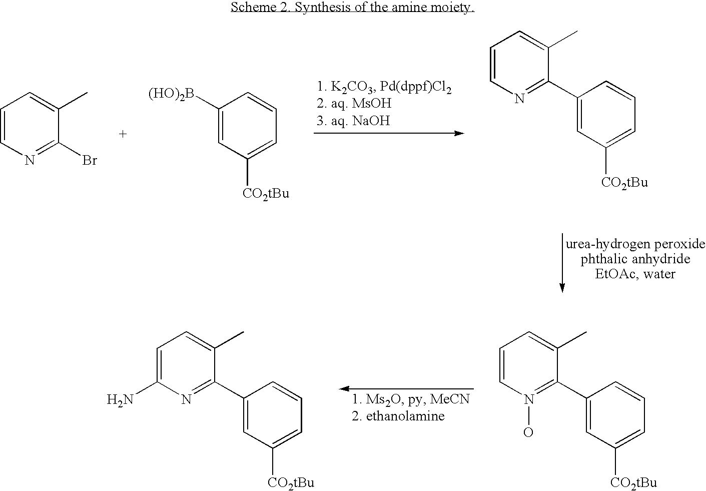

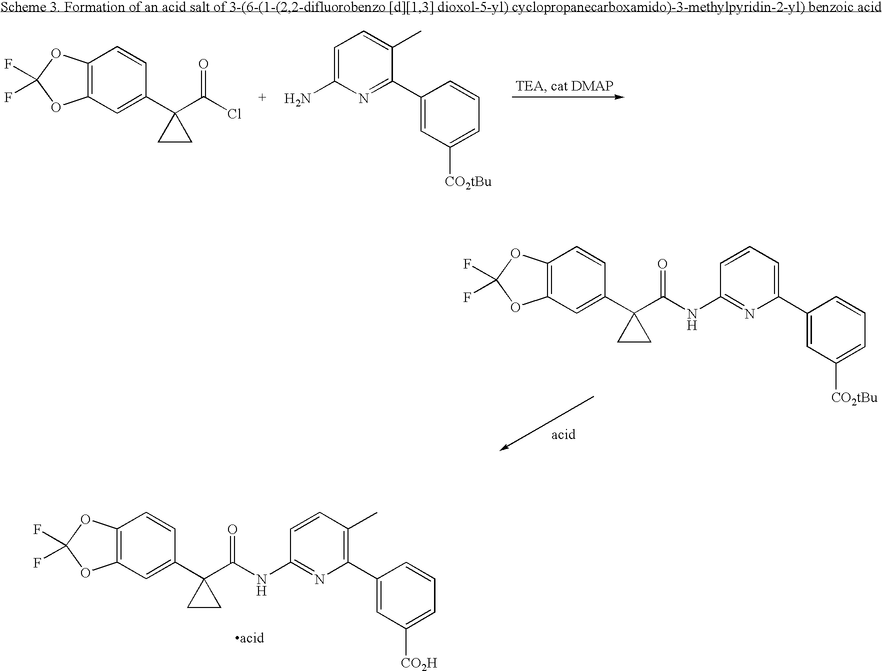

Using the HCl, for example, salt form of 3-(6-(1-(2,2-difluorobenzo[d][1,3]dioxol-5-yl)cyclopropanecarboxamido)-3-methylpyridin-2-yl)benzoic acid as a starting point, Compound 1 can be formed in high yields by dispersing or dissolving the HCl salt form of 3-(6-(1-(2,2-difluorobenzo[d][1,3]dioxol-5-yl)cyclopropanecarboxamido)-3-methylpyridin-2-yl)benzoic acid in an appropriate solvent for an effective amount of time. Other salt forms of 3-(6-(1-(2,2-difluorobenzo[d][1,3]dioxol-5-yl)cyclopropanecarboxamido)-3-methylpyridin-2-yl)benzoic acid may be used such as, for example, other mineral or organic acid forms. The other salt forms result from hydrolysis of the t-butyl ester with the corresponding acid. Other acids/salt forms include nitric, sulfuric, phosphoric, boric, acetic, benzoic, malonic, and the like. The salt form of 3-(6-(1-(2,2-difluorobenzo[d][1,3]dioxol-5-yl)cyclopropanecarboxamido)-3-methylpyridin-2-yl)benzoic acid may or may not be soluble depending upon the solvent used, but lack of solubility does not hinder formation of Compound 1. For example, in one embodiment, the appropriate solvent may be water or an alcohol/water mixture such as an about 50% methanol/water mixture, even though the HCl salt form of 3-(6-(1-(2,2-difluorobenzo[d][1,3]dioxol-5-yl)cyclopropanecarboxamido)-3-methylpyridin-2-yl)benzoic acid is only sparingly soluble in water. In one embodiment, the appropriate solvent is water.

The effective amount of time for formation of Compound 1 from the salt form of 3-(6-(1-(2,2-difluorobenzo[d][1,3]dioxol-5-yl)cyclopropanecarboxamido)-3-methylpyridin-2-yl)benzoic acid can be any time between about 1 and 24 hours. Generally, greater than 24 hours is not needed to obtain high yields (98%), but certain solvents may require greater amounts of time. It is also recognized that the amount of time needed is generally inversely proportional to the temperature. That is, the higher the temperature the less time needed to affect dissociation of acid to form Compound 1. When the solvent is water, stirring the dispersion for approximately 24 hours at room temperature gives Compound 1 in an approximately 98% yield. If a solution of the salt form of 3-(6-(1-(2,2-difluorobenzo[d][1,3]dioxol-5-yl)cyclopropanecarboxamido)-3-methylpyridin-2-yl)benzoic acid is desired for process purposes, an elevated temperature and organic solvent may be used. After stirring the solution for an effective amount of time at the elevated temperature, recrystallization upon cooling yields substantially pure forms of Compound 1. In one embodiment, substantially pure refers to greater than about 90% purity. In another embodiment, substantially pure refers to greater than about 95% purity. In another embodiment, substantially pure refers to greater than about 98% purity. In another embodiment, substantially pure refers to greater than about 99% purity. The temperature selected depends in part on the solvent used and is well within the capabilities of someone of ordinary skill in the art to determine. In one embodiment, the temperature is between room temperature and about 80° C. In another embodiment, the temperature is between room temperature and about 40° C. In another embodiment, the temperature is between about 40° C. and about 60° C. In another embodiment, the temperature is between about 60° C. and about 80° C.

In some embodiments, Compound 1 may be further purified by recrystallization from an organic solvent. Examples of organic solvents include, but are not limited to, toluene, cumene, anisole, 1-butanol, isopropylacetate, butyl acetate, isobutyl acetate, methyl t-butyl ether, methyl isobutyl ketone, or 1-propanol/water (at various ratios). Temperature may be used as described above. For example, in one embodiment, Compound 1 is dissolved in 1-butanol at about 75° C. until it is completely dissolved. Cooling down the solution to about 10° C. at a rate of about 0.2° C./min yields crystals of Compound 1 which may be isolated by filtration.

Uses, Formulation and Administration

Aqueous Formulations

In one aspect of the present invention, aqueous formulations are provided, wherein these formulations comprise Compound 1 as described herein, water, and a viscosity agent, and optionally comprise other agents such as a surfactant, antifoaming agent, taste masker and/or flavorant, and additional pharmaceutically acceptable carriers, adjuvants or vehicles. In certain embodiments, these formulations optionally further comprise one or more additional therapeutic agents.

It will also be appreciated that Compound 1 can exist as a pharmaceutically acceptable derivative or a prodrug thereof. According to the present invention, a pharmaceutically acceptable derivative or a prodrug includes, but is not limited to esters, salts of such esters, or any other adduct or derivative which upon administration to a patient in need thereof is capable of providing, directly or indirectly, a compound as otherwise described herein, or a metabolite or residue thereof.

1. Viscosity Agents

The viscosity agent is chosen from pharmaceutically acceptable viscosity agents, for example xanthan gum, hydroxypropyl cellulose, hydroxypropylmethyl cellulose, methyl cellulose, carageenan, carboxymethyl cellulose, microcrystalline cellulose, polyvinylpyrrolidone, sodium alginate, povidone, acacia, guar gum, tragacanth, magnesium aluminum silicate, and polyacrylates. Preferred viscosity agents comprise methyl cellulose, sodium carboxymethyl cellulose, hydroxypropylmethyl cellulose, hydroxypropyl cellulose, sodium alginate, polyacrylate, povidone, acacia, guar gum, xanthan gum, magnesium aluminum silicate and tragacanth. Particularly preferred viscosity agents are methyl cellulose, polyacrylate, xanthan gum, guar gum, povidone, sodium carboxymethylcellulose, and magnesium aluminum silicate. A particularly preferred viscosity agent is methyl cellulose.

The oral formulations of the present invention generally comprise from about 0.1 to about 20% by weight of viscosity agent. In a preferred embodiment, the concentration of viscosity agent is from about 0.1 to about 1% by weight. In a particularly preferred embodiment, the concentration of viscosity agent is about 0.5% by weight.

2. Surfactants

Surfactants reduce the surface tension between water and an organic compound such as Compound 1 by adsorbing at the water-Compound 1 interface. Surfactants increase the wettability of Compound 1 and contribute to the stability of the aqueous suspension. Surfactants often classified into four primary groups; anionic, cationic, non-ionic, and zwitterionic (dual charge). In a preferred embodiment, the surfactant is an anionic, cationic, or nonionic surfactant.

Anionic surfactants may be chosen from salts of dodecyl sulfate, lauryl sulfate, laureth sulfate, alkyl benzene sulfonates, butanoic acid, hexanoic acid, octanoic acid, decanoic acid, lauric acid, myristic acid, palmitic acid, stearic acid, arachidic acid, behenic acid, myristoleic acid, palmitoleic acid, oleic acid, linoleic acid, alpha-linolenic acid, arachidonic acid, eicosapentaenoic acid, erucic acid, or docosahexaenoic acid.

Cationic surfactants may be chosen from cetyl trimethylammonium bromide, cetylpyridinium chloride, polethoxylated tallow amine, benzalkonium chloride, and benzethonium chloride.

Nonionic surfactants may be chosen from polysorbates, alkyl poly(ethylene oxide), poloxamine, alkyl polyglucosides, octyl glucoside, decyl maltoside, fatty alcohol, cetyl alcohol, oleyl alcohol, cocamide MEA, cocamide DEA, and cocamide TEA. The term “polysorbate” is employed for its art-recognized meaning, i.e., polyoxyethylene sorbitan fatty acid esters as disclosed and defined in the Handbook Of Pharmaceutical Excipients, edited by Ainley Wade and Paul Weller, The Pharmaceutical Press, London, 1994. Useful polysorbates include polysorbate 20, 21, 40, 60, 61, 65, 80, 81, 85, and 120. Polysorbate 80 is preferred. Polysorbate 80 is also commonly referred to as its commercially available trade name “Tween80.”

The oral formulations of the present invention generally comprise from about 0.1 to about 10% by weight surfactant. In a preferred embodiment, the concentration of surfactant is from about 0.1 to about 1% by weight. In a particularly preferred embodiment, the concentration of surfactant is about 0.5% by weight.

3. Antifoaming Agent

As the name suggests, an antifoaming agent is a chemical additive that inhibits the formation of foam. Antifoaming agents are used medicinally in pharmaceutical compositions to relieve bloating because they cause small bubbles to coalesce into large bubbles, which are passed more easily. Many antifoaming agents comprise polydimethylsiloxane. A familiar example is the drug simethicone, which is the active ingredient in drugs such as Gas-X™. Simethicone is a mixture of polydimethylsiloxane and silica gel.

Oral formulations of the present invention generally comprise from about 0.01 to about 0.2% by weight antifoaming agent. In a preferred embodiment, the concentration of antifoaming agent is from about 0.01% to about 0.1% by weight. In a particularly preferred embodiment, the concentration of antifoaming agent is about 0.05% by weight.

4. Buffer

Buffering agents can be either the weak acid or weak base that would comprise a buffer solution. These agents are added to substances that are to be placed into acidic or basic conditions in order to stabilize the substance. Suitable buffers for the oral formulations of the present invention may be chosen from sodium, potassium or ammonium salt of acetic, boric, carbonic, phosphoric, succinic, malic, tartaric, citric, acetic, benzoic, lactic, glyceric, gluconic, glutaric or glutamic acid. In a preferred embodiment, the buffer comprises sodium, potassium or ammonium salts of citric acid.

5. Taste Masker and/or Flavoring Agent

As previously stated, it is advantageous to include a taste masking agent in the oral formulations of Compound 1. Such taste masking agents are alkali metal and alkaline earth metal chlorides including sodium chloride, lithium chloride, potassium chloride, magnesium chloride, and calcium chloride. Sodium chloride is preferred. The taste masking agent is generally included in the suspension in a taste-masking amount, generally an amount of about 0.5 to about 2.0 weight % as taste masker based on the weight of the suspension. For other salts, equivalent molar amounts can be calculated. Other taste maskers include sugars, with or without the presence of other sweetening and/or flavoring agents. When used, flavoring agents may be chosen from synthetic flavor oils and flavoring aromatics and/or natural oils, extracts from plant leaves, flowers, fruits, and so forth and combinations thereof. These may include cinnamon oil, oil of wintergreen, peppermint oils, clove oil, bay oil, anise oil, eucalyptus, thyme oil, cedar leaf oil, oil of nutmeg, oil of sage, oil of bitter almonds, and cassia oil. Also useful as flavors are vanilla, citrus oil, including lemon, orange, grape, lime and grapefruit, and fruit essence, including apple, banana, pear, peach, strawberry, raspberry, cherry, plum, pineapple, apricot, and so forth. The amount of flavoring may depend on a number of factors including the organoleptic effect desired. Generally the flavoring will be present in an amount of from about 0.01 to about 1.0 percent by weight based on the total suspension weight.

As described above, the formulations of the present invention can comprise a pharmaceutically acceptable carrier, adjuvant, or vehicle, additional to water which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, partial glyceride mixtures of saturated vegetable fatty acids, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; alginic acid; pyrogen-free water; isotonic saline; Ringer's solution; and ethyl alcohol, as well as other non-toxic compatible lubricants such as sodium lauryl sulfate and magnesium stearate, as well as coloring agents, releasing agents, coating agents, sweetening, perfuming agents, preservatives and antioxidants can also be present in the composition, according to the judgment of the formulator.

Uses of Compounds and Pharmaceutically Acceptable Compositions

In yet another aspect, the present invention provides a method of treating a condition, disease, or disorder implicated by CFTR. In certain embodiments, the present invention provides a method of treating a condition, disease, or disorder implicated by a deficiency of CFTR activity, the method comprising administering an oral formulation comprising Compound 1 described herein to a subject, preferably a mammal, in need thereof.

A “CFTR-mediated disease” as used herein is a disease selected from cystic fibrosis, Hereditary emphysema, Hereditary hemochromatosis, Coagulation-Fibrinolysis deficiencies, such as Protein C deficiency, Type 1 hereditary angioedema, Lipid processing deficiencies, such as Familial hypercholesterolemia, Type 1 chylomicronemia, Abetalipoproteinemia, Lysosomal storage diseases, such as I-cell disease/Pseudo-Hurler, Mucopolysaccharidoses, Sandhof/Tay-Sachs, Crigler-Najjar type II, Polyendocrinopathy/Hyperinsulemia, Diabetes mellitus, Laron dwarfism, Myleoperoxidase deficiency, Primary hypoparathyroidism, Melanoma, Glycanosis CDG type 1, Hereditary emphysema, Congenital hyperthyroidism, Osteogenesis imperfecta, Hereditary hypofibrinogenemia, ACT deficiency, Diabetes insipidus (DI), Neurophyseal DI, Neprogenic DI, Charcot-Marie Tooth syndrome, Perlizaeus-Merzbacher disease, neurodegenerative diseases such as Alzheimer's disease, Parkinson's disease, Amyotrophic lateral sclerosis, Progressive supranuclear plasy, Pick's disease, several polyglutamine neurological disorders asuch as Huntington, Spinocerebullar ataxia type I, Spinal and bulbar muscular atrophy, Dentatorubal pallidoluysian, and Myotonic dystrophy, as well as Spongiform encephalopathies, such as Hereditary Creutzfeldt-Jakob disease, Fabry disease, Straussler-Scheinker syndrome, COPD, dry-eye disease, and Sjogren's disease.

In certain embodiments, the present invention provides a method of treating a CFTR-mediated disease in a mammal comprising the step of administering to said mammal an effective amount of a composition comprising Compound 1 described herein.

According to an alternative preferred embodiment, the present invention provides a method of treating cystic fibrosis in a human comprising the step of administering to said human an oral formulation comprising Compound 1 described herein.

According to the invention an “effective amount” of an oral formulation of Compound 1 is that amount effective for treating or lessening the severity of any of the diseases recited above.

In certain embodiments, an oral formulation of Compound 1 described herein is useful for treating or lessening the severity of cystic fibrosis in patients who exhibit residual CFTR activity in the apical membrane of respiratory and non-respiratory epithelia. The presence of residual CFTR activity at the epithelial surface can be readily detected using methods known in the art, e.g., standard electrophysiological, biochemical, or histochemical techniques. Such methods identify CFTR activity using in vivo or ex vivo electrophysiological techniques, measurement of sweat or salivary Cl⁻ concentrations, or ex vivo biochemical or histochemical techniques to monitor cell surface density. Using such methods, residual CFTR activity can be readily detected in patients heterozygous or homozygous for a variety of different mutations, including patients homozygous or heterozygous for the most common mutation, ΔF508.

In one embodiment, an oral formulation of Compound 1 described herein is useful for treating or lessening the severity of cystic fibrosis in patients within certain genotypes exhibiting residual CFTR activity, e.g., class III mutations (impaired regulation or gating), class IV mutations (altered conductance), or class V mutations (reduced synthesis) (Lee R. Choo-Kang, Pamela L., Zeitlin, Type I, II, III, IV, and Vcystic fibrosis Tansmembrane Conductance Regulator Defects and Opportunities of Therapy; Current Opinion in Pulmonary Medicine 6:521-529, 2000). Other patient genotypes that exhibit residual CFTR activity include patients homozygous for one of these classes or heterozygous with any other class of mutations, including class I mutations, class II mutations, or a mutation that lacks classification.

In one embodiment, an oral formulation of Compound 1 described herein is useful for treating or lessening the severity of cystic fibrosis in patients within certain clinical phenotypes, e.g., a moderate to mild clinical phenotype that typically correlates with the amount of residual CFTR activity in the apical membrane of epithelia. Such phenotypes include patients exhibiting pancreatic insufficiency or patients diagnosed with idiopathic pancreatitis and congenital bilateral absence of the vas deferens, or mild lung disease.

The exact amount required will vary from subject to subject, depending on the species, age, and general condition of the subject, the severity of the infection, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

In certain embodiments, the compounds of the invention may be administered orally at dosage levels of about 0.01 mg/kg to about 50 mg/kg and preferably from about 1 mg/kg to about 25 mg/kg, of subject body weight per day, one or more times a day, to obtain the desired therapeutic effect.

In certain embodiments, the dosage amount of Compound 1 in the dosage unit form is from about 100 mg to about 1,000 mg. In another embodiment, the dosage amount of Compound 1 is from about 200 mg to about 900 mg. In another embodiment, the dosage amount of Compound 1 is from about 300 mg to about 800 mg. In another embodiment, the dosage amount of Compound 1 is from about 400 mg to about 700 mg. In another embodiment, the dosage amount of Compound 1 is from about 500 mg to about 600 mg.

It will also be appreciated that the oral formulations of Compound 1 described herein can be employed in combination therapies, that is, the oral formulations of Compound 1 can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated”.

In one embodiment, the additional agent is selected from a mucolytic agent, bronchodialator, an anti-biotic, an anti-infective agent, an anti-inflammatory agent, a CFTR modulator other than a compound of the present invention, or a nutritional agent.

In another embodiment, the additional agent is a compound selected from gentamicin, curcumin, cyclophosphamide, 4-phenylbutyrate, miglustat, felodipine, nimodipine, Philoxin B, geniestein, Apigenin, cAMP/cGMP modulators such as rolipram, sildenafil, milrinone, tadalafil, aminone, isoproterenol, albuterol, and almeterol, deoxyspergualin, HSP 90 inhibitors, HSP 70 inhibitors, proteosome inhibitors such as epoxomicin, lactacystin, etc.

In another embodiment, the additional agent is a compound disclosed in WO 2004028480, WO 2004110352, WO 2005094374, WO 2005120497, or WO 2006101740.

In another embodiment, the additiona agent is a benzo[c]quinolizinium derivative that exhibits CFTR modulation activity or a benzopyran derivative that exhibits CFTR modulation activity.

In another embodiment, the addditional agent is a compound disclosed in U.S. Pat. No. 7,202,262, U.S. Pat. No. 6,992,096, US20060148864, US20060148863, US20060035943, US20050164973, WO2006110483, WO2006044456, WO2006044682, WO2006044505, WO2006044503, WO2006044502, or WO2004091502.

In another embodiment, the additional agent is a compound disclosed in WO2004080972, WO2004111014, WO2005035514, WO2005049018, WO2006002421, WO2006099256, WO2006127588, or WO2007044560.

In another embodiment, the additional agent selected from compounds disclosed in U.S. patent application Ser. No. 11/165,818, published as U.S. Published Patent Application No. 2006/0074075, filed Jun. 24, 2005, and hereby incorporated by reference in its entirety. In another embodiment, the additional agent is N-(5-hydroxy-2,4-ditert-butyl-phenyl)-4-oxo-1H-quinoline-3-carboxamide. These combinations are useful for treating the diseases described herein including cystic fibrosis. These combinations are also useful in the kits described herein.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

Toxicology

Summary

The intended clinical route of administration is oral, thus oral toxicity studies were done in mice, rats and dogs. The section Single Dose Toxicity Studies below summarizes the toxicity studies performed: the acute oral toxicity of oral formulations of Compound 1 was assessed in mice and rats given a single dose of Compound 1 followed by a 14-day observation period. Effects of repeat oral dosing were assessed in rats and dogs in preliminary 7-day toxicity studies followed by a 14-day GLP1 toxicity studies. In rats up to 600 mg/kg/day was tolerated without toxic effects. In dogs, up to 200 mg/kg/day was tolerated without toxic effect.

Single oral doses of oral formulations of Compound 1 up to 2000 mg/kg in mice and rats (total dose volume of 20 mL/kg, formulated as a suspension in 0.5% Tween80+0.5% MC in water) resulted in no unscheduled deaths and no significant clinical observations during a 14-day observation period post-dose and were considered well tolerated. There were no effects on organ weights and no gross observations (macroscopic findings) noted at necropsy.

Oral formulations of Compound 1 were well tolerated in both the 7 and 14-day repeat-dose oral toxicity studies. In both rats (dosages up to 600 mg/kg/day) and dogs (up to 200 mg/kg/day), the only findings were mild effects on a few clinical chemistry and hematology parameters at the highest dosage tested. None of these changes was considered adverse and there were no significant Compound 1 related light microscopic lesions in either species. In addition, ECG tracings (dogs) and opthalmology examinations (both species) were all within normal limits. Low prostate: body weight and prostate:brain weight ratios (52-62% at all dose levels) were noted for Compound 1 male dogs vs. the corresponding vehicle control group in the 14-day study. However, in the absence of light microscopic changes in this organ, this was not considered a direct effect of Compound 1, and may have been a spurious result due to the sexual immaturity of the young animals used in this study. The no observed adverse effect level (NOAEL) was thus considered the highest dosage tested in both species: 600 mg/kg/day in rats and 200 mg/kg/day in dogs.

The potential for genetic toxicology was tested using standard GLP bacterial mutation (Ames), Chinese hamster ovary (CHO) chromosome aberration, and in vivo mouse micronucleus assays: Compound 1 was negative in all tests. Data from safety pharmacology studies (ICH S7A/S7B battery) suggest that oral formulations of Compound 1 are unlikely to cause adverse effects on gastrointestinal, respiratory, CNS, or cardiovascular systems in the treatment of CF patients. With the exception of the preliminary 7-day toxicity studies, all studies were performed according to GLP regulations.

In Vitro Studies

Compound 1 was counter screened against a broad panel of enzymes and receptors using radiolabel binding studies (see MDS Pharma Services, LeadProfiling and SpectrumScreen, MDSPS PT#: 1083321). Binding activity was observed only for the Thromboxane A2 (TXA2) receptor (TP receptor) with a Ki of ˜3 μM. In an in vitro functional assay of TP receptor function using rat aorta, Compound 1 was demonstrated to be a TP receptor antagonist with an IC50 between 1 and 10 μM. However, there have been no cardiovascular or respiratory findings in safety pharmacology studies, suggesting that Compound 1 has no TP receptor antagonist effect in vivo, despite achieving very high systemic exposures at oral doses up to 1000 mg/kg in the rat and 200 mg/kg in the dog.

In CF patients, platelet aggregability and TXA2 release is increased, which may contribute to the pathogenesis of bronchoconstriction (O'Sullivan et al., (2005) Blood 105:4635, Stead et al., (1987) Prostaglandins Leukot Med 26:91). The potential TP receptor antagonism of Compound 1 may provide therapeutic benefit in CF patients by preventing TXA2-induced bronchoconstriction.

The effect of Compound 1 on hERG, the cardiac K+ channel responsible for membrane repolarization, was analyzed using a variety of electrophysiological techniques. There was no evidence of a hERG IC50 value below 30 μM in any of these assays, consistent with the lack of hERG channel competitive binding (4% inhibition of 3H-astemizole binding at 10 μM). These findings suggest a low potential for hERG inhibition and its associated QT interval prolongation in vivo.

Single Dose Toxicity Studies

Single oral doses of 500, 1000, or 2000 mg/kg Compound 1 in mice and rats (total dose volume of 20 mL/kg, formulated as a suspension in 0.5% Tween80+0.5% MC in water) resulted in no unscheduled deaths and no significant clinical observations during a 14-day observation period post-dose and were considered well tolerated. There were no effects on organ weights and no gross observations (macroscopic findings) noted at necropsy. Both the maximum tolerated dose(MTD) and no observed adverse effect level (NOAEL) in the acute studies in rats and mice were considered to be >2000 mg/kg.

Mean toxicokinetic parameters at the MTD/NOAEL for both species are summarized in Table 1.

TABLE 1
Mean Values for Selected Non-Compartmental Toxicokinetic.
Parameters for Compound 1 at the MTD/NOAEL for Acute
Oral Toxicity Study in Mice and Rats at 2000 mg/kg.

| | Dose | | AUC_{0-24 h} | Cₘₐₓ | tₘₐₓ |
|---|---|---|---|---|---|
| Species | (mg/kg) | Gender | (μg * hr/mL) | (μg/mL) | (hr) |
| Mice | 2000 | Female | 2899 | 325 | 1 |
| | | Male | 2678 | 264 | 1 |
| Rats | 2000 | Female | 6750 | 305 | 10 |
| | | Male | 6106 | 306 | 24 |

Compound 1 was well absorbed in mice with time to reach maximum plasma concentrations (tₘₐₓ) ranging from 0.5 to 2.0 hr. Maximum plasma concentrations (Cₘₐₓ) and AUC₀₋₂₄ hr increased with increasing dose, but in a less than dose-proportional manner. Cₘₐₓ ranged from 142 mg/mL in males at 500 mg/kg to 325 mg/mL in females at 2000 mg/kg, while AUC₀₋₂₄ₕᵣ ranged from 1837 mg*hr/mL in females at 500 mg/kg to 2899 mg*hr/mL in females at 2000 mg/kg. After reaching the Cₘₐₓ values, Compound 1 concentrations steadily declined from the plasma and the observed elimination half-life (t_{1/2}) values of Compound 1 ranged from 4.1 to 8.6 hr. There were no apparent gender differences in t_{1/2} and t_{1/2} increased with the increase in dose from 500 to 1000 mg/kg. Further increase in dose to 2000 mg/kg did not change the elimination half-life, suggesting saturation of absorption-limited clearance processes. There were no significant gender-related effects in Compound 1 exposures noted.

Compound 1 was also well absorbed in rats with time to reach maximum plasma concentrations(tₘₐₓ) ranging from 4.0 to 24.0 hr. Maximum plasma concentrations (Cₘₐₓ) and AUC₀₋₂₄ₕᵣ increased with increasing dose, but in a less than dose-proportional manner, with the exception of AUC₀₋₂₄ₕᵣ values observed in male rats. Cₘₐₓ ranged from 135 mg/mL in males at 500 mg/kg to 306 mg/mL in males at 2000 mg/kg, while AUC₀₋₂₄ₕᵣ ranged from 1389 mg*hr/mL in males at 500 mg/kg to 6750 mg*hr/mL in females at 2000 mg/kg. After reaching the Cₘₐₓ values, Compound 1 concentrations steadily declined from the plasma and the observed elimination half-life (t_{1/2}) values of Compound 1 ranged from 9.4 to 10.8 hr. The elimination half-life could not be calculated at 1000 and 2000 mg/kg doses of Compound 1. There were no significant gender related effects in Compound 1 exposures noted, with the exception of AUC₀₋₂₄ₕᵣ values in the 500 mg/kg dose group, where more than 2-fold greater values were observed in females vs. males.

Repeat Dose Toxicity Studies

Repeat-dose oral toxicity studies of 7- and 14-days duration have been conducted in rats (up to 600 mg/kg/day) and dogs (up to 200 mg/kg/day).

Compound 1 was well tolerated in the 7-day dose range finding study in rats at dose levels up to 300 mg/kg/day. The animals were dosed orally with the vehicle (0.5% methylcellulose in water), or 15, 75, or 150 mg/kg Compound 1 twice daily for 7 consecutive days. The two daily doses were administered approximately 10 hours apart and the dose volume was 5 mL/kg/b.i.d. for all dose groups. At the end of the dosing period, all animals were euthanized and necropsied. Satellite animals (6/sex/Groups 2-4) were dosed in the same manner as the toxicity animals and plasma samples were collected on Days 1 and 7 for toxicokinetic (TK) analysis. Parameters evaluated during the study were: viability, clinical observations, body weights, feed consumption, clinical pathology (termination), organ weights, macroscopic observations and microscopic pathology. All animals survived until termination of the study.

The plasma AUC₀₋₂₄ data on Day 1 was consistent with previously conducted single dose studies of Compound 1 in the rat. Approximately dose-proportional exposures were observed in both genders at all dose levels on both study days and there were no significant gender effects noted. At the highest dosage (300 mg/kg/day), average plasma concentrations were approximately 260 mM in males (Cₘₐₓ ˜430 mM) and 190 mM in females (Cₘₐₓ ˜280 mM).

Findings in the study were limited to lower serum potassium in the 300 mg/kg/day females and minor effects on body weight in the 300 mg/kg/day animals (both sexes). In addition, males dosed at 300 mg/kg/day Compound 1 had a slight increase in urinary pH and higher adrenal gland weights. These changes were not considered adverse and there were no test article related gross lesions or histopathological findings in any of the tissues examined. Thus, under the conditions of this study, the NOAEL was 300 mg/kg/day.

Similarly, the only findings in the 14-day rat study, at dosages of 150, 300, and 600 mg/kg/day (total dose volume of 5 mL/kg, formulated as a suspension in 0.5% Tween80+0.5% MC in water, given once per day, orally), were mild effects on ALT levels (23-46% increases), total bilirubin (0-54% increases), total cholesterol (21-45% increases), red blood cell (RBC) parameters (total hemoglobin, hematocrit, and RBC counts decreased 4-8%), white blood cell and lymphocyte counts (27-64% increases), and reticulocyte counts (23-32% increases) at the highest dosage only. None of these changes was considered adverse and there were no significant Compound 1 related findings in any of the more than 40 organs and tissues examined by light microscopy. Thus, following 14-days of daily oral administration of Compound 1 to rats, the NOAEL was considered to be 600 mg/kg/day, the highest dosage tested.

Compound 1 was also well tolerated in the 7-day dose range finding study in dogs at dose levels up to 100 mg/kg/day. One dog per gender was dosed orally with the vehicle (0.5% methylcellulose+0.5% Tween80 in water), or 25, 50, or 100 mg/kg/day Compound 1 for 7 consecutive days. The dose volume was 5 mL/kg/day for all dose groups. Plasma samples were collected on Days 1 and 7 for TK analysis and viability, clinical observations, body weights, feed consumption, clinical pathology (termination), organ weights, macroscopic observations, and microscopic pathology were evaluated. All animals survived until termination of the study.

The plasma AUC₀₋₂₄ data on Day 1 was consistent with previously conducted single dose studies of Compound 1 in the dog. Exposures generally increased with increasing dosage in both genders, but the increases were less than dose-proportional and there were no significant gender differences noted. At the highest dosage (100 mg/kg/day), average plasma concentrations were 16 mM in the male (Cₘₐₓ ˜110 mM) and 38 mM in the female (Cₘₐₓ ˜130 mM).

Findings in the study were limited to slight variations in clinical chemistry parameters and a minor effect (0.3 kg loss) on body weight in the 100 mg/kg/day male. These changes were not considered adverse and there were no effects on food consumption, hematologies, coagulations parameters, or ECG measurements, and no test article related gross lesions or histopathological findings in any of the tissues examined. Thus, under the conditions of this study, the NOAEL was 100 mg/kg/day.

Similarly, the only findings in the 14-day dog study, at dosages of 150, 300, and 600 mg/kg/day (total dose volume of 5 mL/kg, formulated as a suspension in 0.5% Tween 80+0.5% MC in water, given once per day, orally), were mild effects on total cholesterol (21-29% decreases), triglycerides (44-46% decreases), and RBC parameters (7-14% decreases). As in the rat studies, these changes were not considered adverse and there were no significant Compound 1 related findings by light microscopy. In addition, ECG tracings and opthalmology examinations were all within normal limits. Low prostate:body weight and prostate:brain weight ratios (52-62% at all dose levels) were noted for all Compound 1 males vs. the corresponding vehicle control group in the 14-day dog study. However, in the absence of light microscopic changes in this organ, this was not considered a direct effect of Compound 1, and may have been a spurious result due to the sexual immaturity of the young animals used in this study. There were no significant, test article-related findings in any of the organs and tissues examined by light microscopy from either of the dog or rat 14-d studies. Thus, following 14-days of daily oral administration of Compound 1 to dogs, the NOAEL was considered to be 200 mg/kg/day, the highest dosage tested.

Mean toxicokinetic parameters at the NOAEL for both species are summarized in Table 2. Systemic exposures in the repeat-dose toxicity studies were high and sustained throughout the dosing duration: at the respective NOAELs, average Cₘₐₓ and AUC₀₋₂₄ₕᵣ values were 222 μg/mL and 3951 μg*hr/mL in rats, and 75 mg/mL and 645 μg*hr/mL in dogs. There were no major differences noted in TK parameters between males and females, or when comparing study Day 1 to study Day 14.

TABLE 2
Mean Values for Selected Non-Compartmental Toxicokinetic
Parameters for Compound 1 at the NOAEL in 14-Day Oral Toxicity
Studies in Rats (600 mg/kg/day) and Dogs (200 mg/kg/day).

| | Study | Dosage | | AUC_{0-24 hr} | Cₘₐₓ | Tₘₐₓ |
|---|---|---|---|---|---|---|
| Species | Day | (mg/kg/day) | Gender | (μg * hr/mL) | (μg/mL) | (hr) |
| Rat | 1 | 600 | Female | 4427 | 273 | 4 |
| | | | Male | 2657 | 164 | 10 |
| | 14 | 600 | Female | 5501 | 272 | 10 |
| | | | Male | 3219 | 181 | 10 |
| Dog | 1 | 200 | Female | 573 | 56 | 4 |
| | | | Male | 691 | 77 | 3 |
| | 14 | 200 | Female | 720 | 77 | 3 |
| | | | Male | 598 | 72 | 3 |

Genotoxicity

Compound 1 did not induce a significant increase in reverse mutations in the bacterial mutation (Ames) assay, and was negative for clastogenicity (chromosome aberrations) in the Chinese hamster ovary (CHO) cell assay. Compound 1 did not induce a significant increase in the number of micronucelated polychromatic erythrocytes when administered to male mice (in vivo mammalian micronucleus assay) by oral gavage at doses up to 2000 mg/kg.

Discussion and Conclusion

Oral formulations of Compound 1 were well tolerated in acute toxicity studies in mice and rats, and in repeat dose toxicity studies in rats and dogs. No genotoxicity liabilities have been found. The NOAEL in 14-day, repeat-dose toxicology studies was at least 600 mg/kg/day in the rat and 200 mg/kg/day in the dog. Basing the calculation on body surface area, the human equivalent dose is at least 95 mg/kg using either NOAEL. Assuming a 60 kg human, this would equate to a total daily dose of approximately 5700 mg.

Safety margin calculations are based on NOAELs and doses to achieve predicted efficacious plasma levels, which are not adjusted for lung distribution or plasma protein binding. Assuming the efficacious dose in rats is 2.3 mg/kg b.i.d., based on Ctrough targets to achieve and maintain the EC90 level, the projected safety margin from the rat NOAEL is 130×. Assuming the efficacious dose in dogs is 0.91 mg/kg b.i.d., based on Ctrough target to achieve and maintain EC90 levels, the projected safety margin range from the dog NOAEL is believed to be 110×.

Pharmacokinetics and Drug Metabolism

Summary

The pharmacokinetics of Compound 1 were assessed in the same species used in the toxicology studies: CD-1 mice, Sprague Dawley rats and beagle dogs. The pharmacokinetics of Compound 1 were also assessed in cynomolgus monkeys. Two crystalline forms of 3-(6-(1-(2,2-difluorobenzo[d][1,3]dioxol-5-yl)cyclopropanecarboxamido)-3-methylpyridin-2-yl)benzoic acid, the free form (Compound 1) and the HCl salt, were used for toxicology and pharmacokinetic studies.

The absorption of Compound 1 in the rat from a methylcellulose suspension is excellent, ranging from 47% to 100%. The bioavailability of Compound 1 in the dog is 53% at 10 mg/kg and 20% at 200 mg/kg when administered orally in a methylcellulose suspension. Compound 1 has very low clearance in the rat, mouse, dog and monkey. The half-life of Compound 1 when administered orally to rats or dogs is 5 to 9 hours. The systemic exposure to Compound 1 in rats in a methylcellulose suspension is proportional to the dose administered across the 1 to 300 mg/kg nominal dose range. In dogs the systemic exposure to 3-(6-(1-(2,2-difluorobenzo[d][1,3]dioxol-5-yl)cyclopropanecarboxamido)-3-methylpyridin-2-yl)benzoic acid administered orally as the HCl salt is proportional to the dose administered across a dose range of 1 to 200 mg/kg.

Following a single oral administration of unlabelled Compound 1 to rats, the highest distribution was to the liver followed by lung, pancreas and brain with tissue-to-plasma ratios of 0.73, 0.19, 0.13 and 0.02, respectively, at one hour following oral administration of 75 mg/kg. Elimination of Compound 1 was nearly complete from all tissues 48 hours after administration (concentrations less than 1 μg/mL). Although the distribution to the lung is low relative to plasma, the concentrations measured in lung at low and moderate doses are predicted to be efficacious.

Absorption

The absorption of 3-(6-(1-(2,2-difluorobenzo[d][1,3]dioxol-5-yl)cyclopropanecarboxamido)-3-methylpyridin-2-yl)benzoic acid was assessed in rats and dogs following oral administration of Compound 1 or HCl salt in methylcellulose suspension formulations. Non-validated research grade assays were used for these analyses. All pharmacokinetic studies were conducted using fed animals unless otherwise specified.

In male Sprague Dawley rats orally administered Compound 1 in a methylcellulose suspension, dose-proportional exposure was observed over the dose range of 1 to 300 mg/kg, as measured by plasma AUC_{0-INF} and Cₘₐₓ values (Table 3). At 600 mg/kg, the increase in systemic exposure was less than proportional to the increase in dose. Bioavailability ranged from 47% to ˜100% across the 1 to 600 mg/kg oral dose range in rats, indicating excellent absorption of the compound.

Terminal half-lives of 5.9 to 8.1 hours were measured over the 1 to 600 mg/kg oral dose range. The tₘₐₓ values ranged from 3.0 to 4.7 hours across the oral dose range studied in rats.

TABLE 3
Mean (SD) Pharmacokinetic Parameters for Compound 1 Following
Single Oral Administration of the Free Form in Suspension to Male Rats.

| Nominal | | | | | | | |
|---|---|---|---|---|---|---|---|
| Dose | AUC_{0-INF} | Cₘₐₓ | tₘₐₓ | t_{1/2} | F | Cₗₐₛₜ | tₗₐₛₜ |
| (mg/kg) | (hr · μg/mL) | (μg/mL) | (hr) | (hr) | (%) | (ng/mL) | (hr) |
| 1 | 13.7 (7.1) | 1.10 | 3.00 | 7.65 | >100 | 28.8 | 48 |
| | | (0.54) | (2.65) | (0.38) | | (11.5) | |
| 30 | 369 (97) | 28.2 | 4.00 | 6.83 | >100 | 44.0 | 48 |
| | | (3.50) | (2.00) | (1.15) | | (55.0) | |
| 150 | 728 (332) | 54.7 | 4.67 | 8.07 | 47 | 51.6 | 72 |
| | | (30.9) | (1.15) | (1.82) | | (26.9) | |
| 300 | 2530 (99) | 181 | 3.33 | 5.86 | 81 | 1120 | 48 |
| | | (220) | (1.15) | (1.22) | | (572) | |
| 600 | 3085 (493) | 203 | 4.67 | 7.49 | 50 | 1236 | 72 |
| | | (35) | (1.15) | (1.11) | | (1234) | |

The oral pharmacokinetics of the HCl salt of 3-(6-(1-(2,2-difluorobenzo[d][1,3]dioxol-5-yl)cyclopropanecarboxamido)-3-methylpyridin-2-yl)benzoic acid were evaluated under fed and fasted conditions in male Sprague Dawley rats at a dose of 30 mg/kg (Table 4). The systemic exposure to 3-(6-(1-(2,2-difluorobenzo[d][1,3]dioxol-5-yl)cyclopropanecarboxamido)-3-methylpyridin-2-yl)benzoic acid.HCl following administration of the HCl salt was similar under fed and fasted conditions, and was similar to the exposure obtained for Compound 1 at the same dose level under fed conditions. The Cₘₐₓ under fed conditions (36.1 μg/mL) was lower than that under fasted conditions (52.3 μg/mL), possibly due to decreased gastric emptying time in the presence of food. The tₘₐₓ following oral administration of the HCl salt was 3.7 hours under fed and 3.3 hours under fasted conditions, which was similar to the tₘₐₓ of 3.0 to 4.7 hours following oral administration of Compound 1 under fed conditions. The terminal half-life of 3-(6-(1-(2,2-difluorobenzo[d][1,3]dioxol-5-yl)cyclopropanecarboxamido)-3-methylpyridin-2-yl)benzoic acid was 5.9 to 8.1 hours (Compound 1) or 5.4 to 6.1 hours (HCl salt) in male rats following oral administration.

TABLE 4
Mean (SD) Pharmacokinetic Parameters for Compound 1 Following
Single Oral Administration of the HCl Salt in Suspension to Male Rats
Under Fed and Fasted.

| Nominal | | | | | | | | |
|---|---|---|---|---|---|---|---|---|
| Dose | | AUC_{0-INF} | Cₘₐₓ | tₘₐₓ | t_{1/2} | F | Cₗₐₛₜ | tₗₐₛₜ |
| (mg/kg) | Cond. | (hr · μg/mL) | (μg/mL) | (hr) | (hr) | (%) | (ng/mL) | (hr) |
| 30 | Fasted | 383 | 52.3 | 3.33 | 5.36 | >100 | 60 | 48 |
| | | (41) | (3.9) | (1.15) | (0.98) | | (18) | |
| 30 | Fed | 340 | 36.1 | 3.67 | 6.07 | >100 | 240 | 48 |
| | | (137) | (10.2) | (2.52) | (0.67) | | (202) | |

The pharmacokinetic parameters for 3-(6-(1-(2,2-difluorobenzo[d][1,3]dioxol-5-yl)cyclopropanecarboxamido)-3-methylpyridin-2-yl)benzoic acid following single oral administration in suspension to male beagle dogs are presented in Table 5.3-(6-(1-(2,2-difluorobenzo[d][1,3]dioxol-5-yl)cyclopropanecarboxamido)-3-methylpyridin-2-yl)benzoic acid (HCl salt) was initially administered at nominal dose levels of 1 to 10 mg/kg in 0.5% methylcellulose/water, however, due to variability in exposure with this vehicle, 0.5% polysorbate 80 was added to the formulation for higher dose levels. At nominal dose levels of 5 to 200 mg/kg in 0.5% polysorbate 80/0.5% methylcellulose/water, exposure was high and relatively dose proportional to 200 mg/kg (Table 5). The half-life ranged from 4.9 to 8.8 hours for all dose levels studied in both vehicles. Bioavailability ranged from 24% to 49% at all dose levels studied and in both suspension formulations.

TABLE 5
Mean (SD) Pharmacokinetic Parameters for 3-(6-(1-(2,2-
difluorobenzo[d][1,3]dioxol-5-yl) cyclopropanecarboxamido)-
3-methylpyridin-2-yl)benzoic acid (HCl Salt) Following Single
Oral Administration in Suspension to Male Dogs.

| | Nomi- | | | | | |
|---|---|---|---|---|---|---|
| | nal | | | | | |
| | Dose | AUC_{0-INF} | Cₘₐₓ | T_{1/2} | Tₘₐₓ | F |
| Vehicle | (mg/kg) | (hr · μg/mL) | (μg/mL) | (hr) | (hr) | (%) |
| 0.5% MC/ | 1 | 2.2 (0.5) | 0.37 (0.2) | 6.0 | 0.83 | 35 |
| Water | 3 | 9.3 (4.8) | 1.1 (0.5) | 5.3 | 1.7 | 49 |
| | 10 | 28.4 (12.8) | 8.9 (7.2) | 8.8 | 1.0 | 40 |
| 0.5% | 5 | 11.0 (3.6) | 3.0 (0.5) | 7.0 | 1.0 | 24 |
| polysorbate | 25 | 56.8 (22.1) | 18.0 (3.7) | 5.9 | 2.0 | 41 |
| 80/0.5% | 50 | 63.4 (22.9) | 21.0 (7.1) | 4.9 | 1.7 | 27 |
| MC/Water | 100 | 268 (123) | 54.4 (6.8) | 5.4 | 1.7 | 46 |
| | 200 | 523 (46.8) | 101 (30.2) | 5.9 | 2.3 | 30 |

The oral pharmacokinetics of Compound 1 were determined under fed and fasted conditions in male beagle dogs following a single 10 mg/kg administration (Table 6). Plasma AUC_{0-INF} of Compound 1 was comparable in the fasted or fed state, although the Cₘₐₓ under fasted conditions (7.9 μg/mL) was higher than under fed conditions (4.1 μg/mL). The tₘₐₓ occurred sooner after administration in the fasted state (1.5 hours) than in the fed state (2.7 hours). The variability of the systemic exposure of Compound 1 was higher under fed conditions (CV of 67% for AUC_{0-INF}) than under fasted conditions (CV of 27% for AUC_{0-INF}), possibly due to changes in stomach emptying time under fed conditions.

TABLE 6
Mean (SD) Pharmacokinetic Parameters for Compound 1 Following
Oral Administration to Fed or Fasted Male Dogs at 10 mg/kg.

| | Nomi- | | | | | | | |
|---|---|---|---|---|---|---|---|---|
| | nal | AUC_{0-INF} | Cₘₐₓ | | | | Cₗₐₛₜ | |
| | Dose | (hr · | (μg/ | tₘₐₓ | t_{1/2} | F | (ng/ | tₗₐₛₜ |
| Cond. | (mg/kg) | μg/mL) | mL) | (hr) | (hr) | (%) | mL) | (hr) |
| Fasted | 10 | 35.8 | 7.9 | 1.5 | 6.3 | 53 | 66 | 48 |
| | | (9.7) | (1.9) | (0.6) | (3.3) | | (36) | |
| Fed | 10 | 35.7 | 4.1 | 2.7 | 6.6 | 53 | 47 | 48 |
| | | (24) | (1.2) | (0.8) | (4.4) | | (34) | |

Following oral administration of Compound 1 and HCl salt of 3-(6-(1-(2,2-difluorobenzo[d][1,3]dioxol-5-yl)cyclopropanecarboxamido)-3-methylpyridin-2-yl)benzoic acid to male dogs at 200 mg/kg, higher systemic AUC_{0-INF} and Cₘₐₓ values were observed for the HCl salt (755 μg·hr/mL and 133 μg/mL, respectively) than for Compound 1 (288 μg·hr/mL and 52 μg/mL, respectively) (Table 7). The tₘₐₓ and t_{1/2} for 3-(6-(1-(2,2-difluorobenzo[d][1,3]dioxol-5-yl)cyclopropanecarboxamido)-3-methylpyridin-2-yl)benzoic acid were similar after administration of Compound 1 and HCl salt (Table 7).

TABLE 7
Mean (SD) Pharmacokinetic Parameters for 3-(6-(1-(2,2-
difluorobenzo[d][1,3]dioxol-5-yl) cyclopropanecarboxamido)-
3-methylpyridin-2-yl)benzoic acid Following Oral Administration of
Compound 1 and HCl Salt in Suspension to Male Dogs at 200 mg/kg.

| | Nomi- | | | | | | | |
|---|---|---|---|---|---|---|---|---|
| | nal | AUC_{0-INF} | Cₘₐₓ | | | | | |
| | Dose | (hr · | (μg/ | tₘₐₓ | t_{1/2} | F | Cₗₐₛₜ | tₗₐₛₜ |
| Form | (mg/kg) | μg/mL) | mL) | (hr) | (hr) | (%) | (ng/mL) | (hr) |
| Free | 200 | 288 | 52 | 1.7 | 6.1 | 20 | 63 | 48 |
| | | (306) | (38) | (1.2) | (3.2) | | (66) | |
| HCL | 200 | 755 | 133 | 2.5 | 5.5 | 53 | 128 | 48 |
| salt | | (114) | (26) | (0.6) | (2.4) | | (147) | |

Distribution

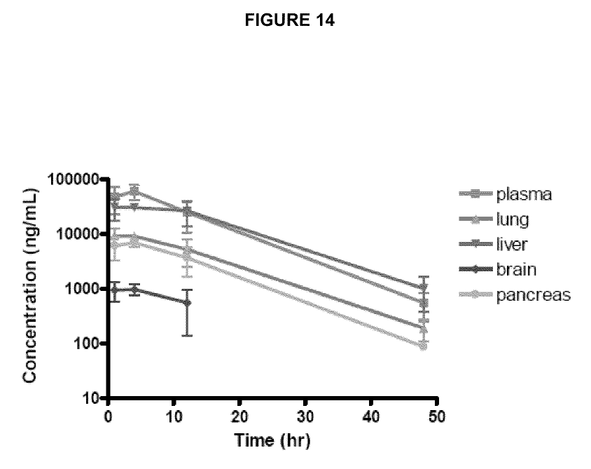

Following oral administration to rats at 75 mg/kg in 0.5% methylcellulose/water suspension, tissue-to-plasma concentration ratios of 3-(6-(1-(2,2-difluorobenzo[d][1,3]dioxol-5-yl)cyclopropanecarboxamido)-3-methylpyridin-2-yl)benzoic acid at 1 to 48 hours after administration were highest in liver (0.7 to 1.8), then lung (0.15 to 0.35), pancreas (0.12 to 0.15), and lowest in brain (0.02) (Table 8, FIG. 14). At 48 hours the concentrations of 3-(6-(1-(2,2-difluorobenzo[d][1,3]dioxol-5-yl)cyclopropanecarboxamido)-3-methylpyridin-2-yl)benzoic acid were extremely low (less than 1 μg/mL) indicating near complete elimination from tissues. The rates of elimination of 3-(6-(1-(2,2-difluorobenzo[d][1,3]dioxol-5-yl)cyclopropanecarboxamido)-3-methylpyridin-2-yl)benzoic acid from all tissues measured were similar to its plasma elimination rate (Table 9).

TABLE 8
Mean Tissue Concentrations and Tissue/Plasma Ratios of 3-(6-(1-
(2,2-difluorobenzo[d][1,3]dioxol-5-yl) cyclopropanecarboxamido)-
3-methylpyridin-2-yl)benzoic acid in Male Rats Following Single
Oral Administration at a Dose of 75 mg/kg.

| | Tissue Concentrations | | | | | | | | |
|---|---|---|---|---|---|---|---|---|---|
| | (μg/mL or μg/g) | | | | | Tissue/Plasma Ratio | | | |
| Time | | | | Pan- | | | | Pan- | |
| (hr) | Plasma | Liver | Lung | creas | Brain | Liver | Lung | creas | Brain |
| 1 | 47.3 | 34.7 | 9.14 | 6.03 | 0.93 | 0.73 | 0.19 | 0.13 | 0.02 |
| 4 | 59.1 | 38.1 | 8.99 | 6.83 | 0.96 | 0.64 | 0.15 | 0.12 | 0.02 |
| 12 | 24.8 | 26.3 | 5.17 | 3.65 | 0.55 | 1.06 | 0.21 | 0.15 | 0.02 |
| 48 | 0.54 | 0.97 | 0.19 | 0.09 | BLQ | 1.78 | 0.35 | 0.15 | BLQ |

TABLE 9
Summary of Tissue Pharmacokinetic Parameters of 3-(6-(1-(2,2-
difluorobenzo[d][1,3]dioxol-5-yl) cyclopropanecarboxamido)-
3-methylpyridin-2-yl)benzoic acid in Male Sprague Dawley Rats
Following Single Oral Administration at a Dose of 75 mg/kg.

| | | | | | | | Cₘₐₓ |
|---|---|---|---|---|---|---|---|
| | AUC_{0-INF} | | | | | | Ratio |
| | (hr · | Cₘₐₓ | tₘₐₓ | Cₗₐₛₜ | tₗₐₛₜ | t_{1/2} | (Tissue/ |
| Tissue | μg/mL) | (μg/mL) | (hr) | (ng/mL) | (hr) | (hr) | Plasma) |
| Plasma | 980266 | 59067 | 4 | 542 | 48 | 6.15 | — |
| Liver | 885161 | 38067 | 4 | 965 | 48 | 8.06 | 0.64 |
| Lung | 187039 | 9140 | 1 | 189 | 48 | 7.79 | 0.15 |
| Pancreas | 132270 | 6833 | 4 | 86 | 48 | 6.87 | 0.12 |
| Brain | 9304^{a} | 957 | 4 | 546 | 12 | — | 0.02 |

^{a}AUCₐₗₗ.

In order that the invention described herein may be more fully understood, the following examples are set forth. It should be understood that these examples are for illustrative purposes only and are not to be construed as limiting this invention in any manner.

### EXAMPLE

### Methods & Materials

Differential Scanning Calorimetry (DSC)

The Differential scanning calorimetry (DSC) data of Form I were collected using a DSC Q100 V9.6 Build 290 (TA Instruments, New Castle, Del.). Temperature was calibrated with indium and heat capacity was calibrated with sapphire. Samples of 3-6 mg were weighed into aluminum pans that were crimped using lids with 1 pin hole. The samples were scanned from 25° C. to 350° C. at a heating rate of 10° C./min and with a nitrogen gas purge of 50 ml/min. Data were collected by Thermal Advantage Q Series™ version 2.2.0.248 software and analyzed by Universal Analysis software version 4.1D (TA Instruments, New Castle, Del.). The reported numbers represent single analyses.

XRPD (X-ray Powder Diffraction)

The X-Ray diffraction (XRD) data of Compound 1 were collected on a Bruker D8 DISCOVER powder diffractometer with HI-STAR 2-dimensional detector and a flat graphite monochromator. Cu sealed tube with Kα radiation was used at 40 kV, 35 mA. The samples were placed on zero-background silicon wafers at 25° C. For each sample, two data frames were collected at 120 seconds each at 2 different θ₂ angles: 8° and 26°. The data were integrated with GADDS software and merged with DIFFRACT^{plus}EVA software. Uncertainties for the reported peak positions are ±0.2 degrees.

Vitride® (sodium bis(2-methoxyethoxy)aluminum hydride [or NaAlH₂(OCH₂CH₂OCH₃)₂], 65 wgt % solution in toluene) was purchased from Aldrich Chemicals.

2,2-Difluoro-1,3-benzodioxole-5-carboxylic acid was purchased from Saltigo (an affiliate of the Lanxess Corporation).

Anywhere in the present application where a name of a compound may not correctly describe the structure of the compound, the structure supersedes the name and governs.

### Synthesis of 3-(6-(1-(2,2-difluorobenzo[d][1,3]dioxol-5-yl)cyclopropanecarboxamido)-3-methylpyridin-2-yl)benzoic acid.HCl

Acid Chloride Moiety

### Synthesis of (2,2-difluoro-1,3-benzodioxol-5-yl)-methanol

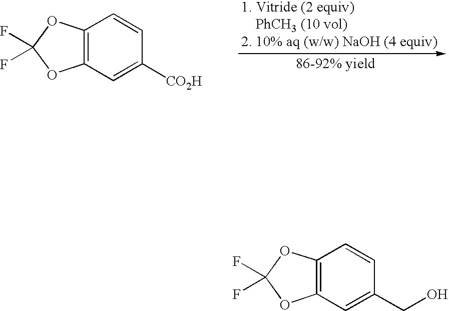

Commercially available 2,2-difluoro-1,3-benzodioxole-5-carboxylic acid (1.0 eq) is slurried in toluene (10 vol). Vitride® (2 eq) is added via addition funnel at a rate to maintain the temperature at 15-25° C. At the end of addition the temperature is increased to 40° C. for 2 h then 10% (w/w) aq. NaOH (4.0 eq) is carefully added via addition funnel maintaining the temperature at 40-50° C. After stirring for an additional 30 minutes, the layers are allowed to separate at 40° C. The organic phase is cooled to 20° C. then washed with water (2×1.5 vol), dried (Na₂SO₄), filtered, and concentrated to afford crude (2,2-difluoro-1,3-benzodioxol-5-yl)-methanol that is used directly in the next step.

### Synthesis of 5-chloromethyl-2,2-difluoro-1,3-benzodioxole

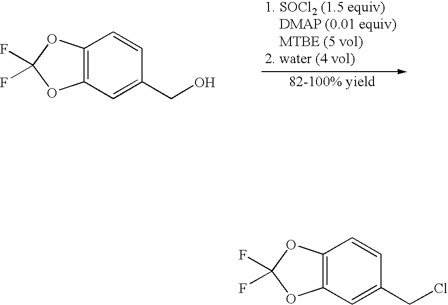

(2,2-difluoro-1,3-benzodioxol-5-yl)-methanol (1.0 eq) is dissolved in MTBE (5 vol). A catalytic amount of DMAP (1 mol %) is added and SOCl₂ (1.2 eq) is added via addition funnel. The SOCl₂ is added at a rate to maintain the temperature in the reactor at 15-25° C. The temperature is increased to 30° C. for 1 hour then cooled to 20° C. then water (4 vol) is added via addition funnel maintaining the temperature at less than 30° C. After stirring for an additional 30 minutes, the layers are allowed to separate. The organic layer is stirred and 10% (w/v) aq. NaOH (4.4 vol) is added. After stirring for 15 to 20 minutes, the layers are allowed to separate. The organic phase is then dried (Na₂SO₄), filtered, and concentrated to afford crude 5-chloromethyl-2,2-difluoro-1,3-benzodioxole that is used directly in the next step.

### Synthesis of (2,2-difluoro-1,3-benzodioxol-5-yl)-acetonitrile

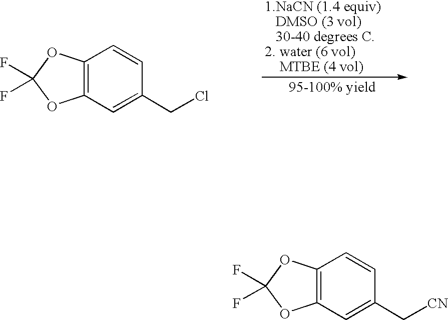

A solution of 5-chloromethyl-2,2-difluoro-1,3-benzodioxole (1 eq) in DMSO (1.25 vol) is added to a slurry of NaCN (1.4 eq) in DMSO (3 vol) maintaining the temperature between 30-40° C. The mixture is stirred for 1 hour then water (6 vol) is added followed by MTBE (4 vol). After stirring for 30 min, the layers are separated. The aqueous layer is extracted with MTBE (1.8 vol). The combined organic layers are washed with water (1.8 vol), dried (Na₂SO₄), filtered, and concentrated to afford crude (2,2-difluoro-1,3-benzodioxol-5-yl)-acetonitrile (95%) that is used directly in the next step.

### Synthesis of (2,2-difluoro-1,3-benzodioxol-5-yl)-cyclopropanecarbonitrile

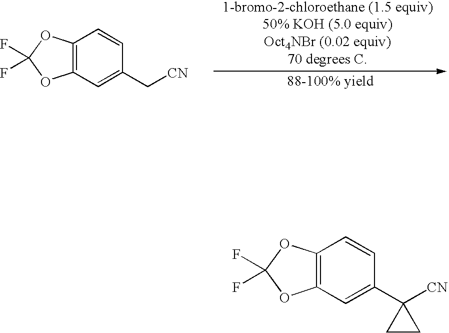

A mixture of (2,2-difluoro-1,3-benzodioxol-5-yl)-acetonitrile (1.0 eq), 50 wt % aqueous KOH (5.0 eq) 1-bromo-2-chloroethane (1.5 eq), and Oct₄NBr (0.02 eq) is heated at 70° C. for 1 h. The reaction mixture is cooled then worked up with MTBE and water. The organic phase is washed with water and brine then the solvent is removed to afford (2,2-difluoro-1,3-benzodioxol-5-yl)-cyclopropanecarbonitrile.

### Synthesis of 1-(2,2-difluoro-1,3-benzodioxol-5-yl)-cyclopropanecarboxylic acid

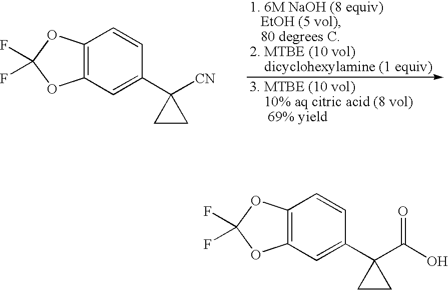

(2,2-difluoro-1,3-benzodioxol-5-yl)-cyclopropanecarbonitrile is hydrolyzed using 6 M NaOH (8 equiv) in ethanol (5 vol) at 80° C. overnight. The mixture is cooled to room temperature and ethanol is evaporated under vacuum. The residue is taken into water and MTBE, 1 M HCl was added and the layers are separated. The MTBE layer was then treated with dicyclohexylamine (0.97 equiv). The slurry is cooled to 0° C., filtered and washed with heptane to give the corresponding DCHA salt. The salt is taken into MTBE and 10% citric acid and stirred until all solids dissolve. The layers are separated and the MTBE layer was washed with water and brine. Solvent swap to heptane followed by filtration gives 1-(2,2-difluoro-1,3-benzodioxol-5-yl)-cyclopropanecarboxylic acid after drying in a vacuum oven at 50° C. overnight.

### Synthesis of 1-(2,2-difluoro-1,3-benzodioxol-5-yl)-cyclopropanecarbonyl chloride

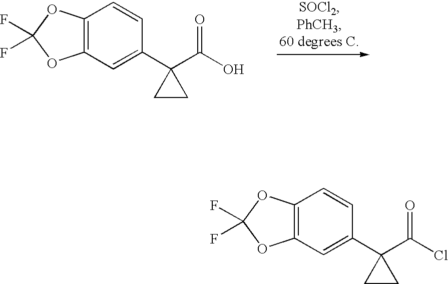

1-(2,2-difluoro-1,3-benzodioxol-5-yl)-cyclopropanecarboxylic acid (1.2 eq) is slurried in toluene (2.5 vol) and the mixture heated to 60° C. SOCl₂ (1.4 eq) is added via addition funnel. The toluene and SOCl₂ are distilled from the reaction mixture after 30 minutes. Additional toluene (2.5 vol) is added and distilled again.

Amine Moiety

### Synthesis of tert-butyl-3-(3-methylpyridin-2-yl)benzoate

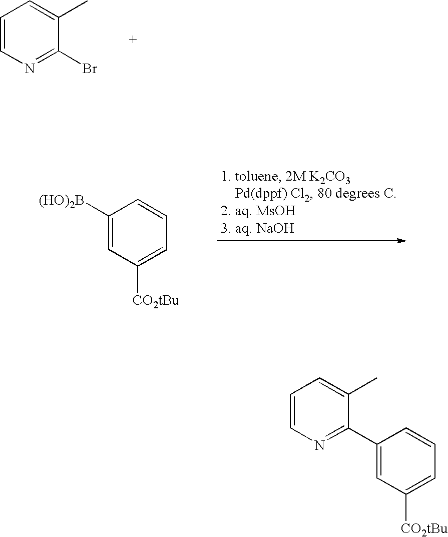

2-Bromo-3-methylpyridine (1.0 eq) is dissolved in toluene (12 vol). K₂CO₃ (4.8 eq) is added followed by water (3.5 vol) and the mixture heated to 65° C. under a stream of N₂ for 1 hour. 3-(t-Butoxycarbonyl)phenylboronic acid (1.05 eq) and Pd(dppf)Cl₂.CH₂Cl₂ (0.015 eq) are then added and the mixture is heated to 80° C. After 2 hours, the heat is turned off, water is added (3.5 vol) and the layers are allowed to separate. The organic phase is then washed with water (3.5 vol) and extracted with 10% aqueous methanesulfonic acid (2 eq MsOH, 7.7 vol). The aqueous phase is made basic with 50% aqueous NaOH (2 eq) and extracted with EtOAc (8 vol). The organic layer is concentrated to afford crude tert-butyl-3-(3-methylpyridin-2-yl)benzoate (82%) that is used directly in the next step.

### Synthesis of 2-(3-(tert-butoxycarbonyl)phenyl)-3-methylpyridine-1-oxide

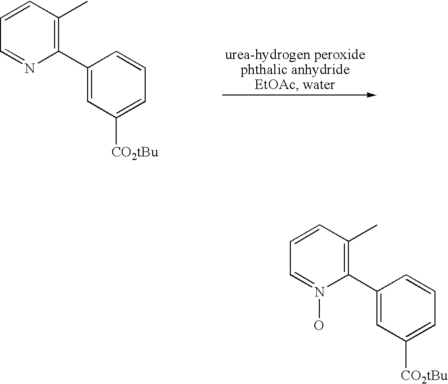

tert-Butyl-3-(3-methylpyridin-2-yl)benzoate (1.0 eq) is dissolved in EtOAc (6 vol). Water (0.3 vol) is added followed by urea-hydrogen peroxide (3 eq). The phthalic anhydride (3 eq) is added portion-wise as a solid to maintain the temperature in the reactor below 45° C. After completion of phthalic anhydride addition, the mixture is heated to 45° C. After stirring for an additional 4 hours, the heat is turned off. 10% w/w aqueous Na₂SO₃ (1.5 eq) is added via addition funnel. After completion of Na₂SO₃ addition, the mixture is stirred for an additional 30 minutes and the layers separated. The organic layer is stirred and 10% w/w aq. Na₂CO₃ (2 eq) is added. After stirring for 30 minutes, the layers are allowed to separate. The organic phase is washed 13% w/v aq NaCl. The organic phase is then filtered and concentrated to afford crude 2-(3-(tert-butoxycarbonyl)phenyl)-3-methylpyridine-1-oxide (95%) that is used directly in the next step.

### Synthesis of tert-butyl-3-(6-amino-3-methylpyridin-2-yl)benzoate

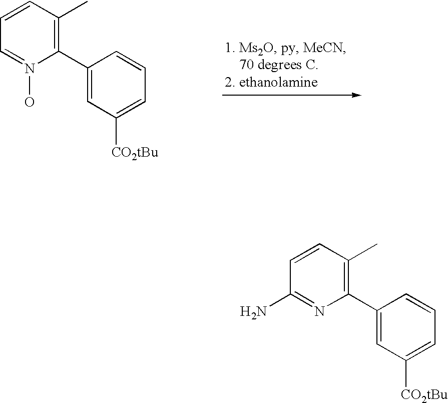

A solution of 2-(3-(tert-butoxycarbonyl)phenyl)-3-methylpyridine-1-oxide (1 eq) and pyridine (4 eq) in MeCN (8 vol) is heated to 70° C. A solution of methanesulfonic anhydride (1.5 eq) in MeCN (2 vol) is added over 50 min via addition funnel maintaining the temperature at less than 75° C. The mixture is stirred for an additional 0.5 hours after complete addition. The mixture is then allowed to cool to ambient. Ethanolamine (10 eq) is added via addition funnel. After stirring for 2 hours, water (6 vol) is added and the mixture is cooled to 10° C. After stirring for NLT 3 hours, the solid is collected by filtration and washed with water (3 vol), 2:1 MeCN/water (3 vol), and MeCN (2×1.5 vol). The solid is dried to constant weight (<1% difference) in a vacuum oven at 50° C. with a slight N₂ bleed to afford tert-butyl-3-(6-amino-3-methylpyridin-2-yl)benzoate as a red-yellow solid (53% yield).

### Synthesis of 3-(6-(1-(2,2-difluorobenzo[d][1,3]dioxol-5-yl)cyclopropanecarboxamido)-3-methylpyridin-2-yl)-t-butylbenzoate

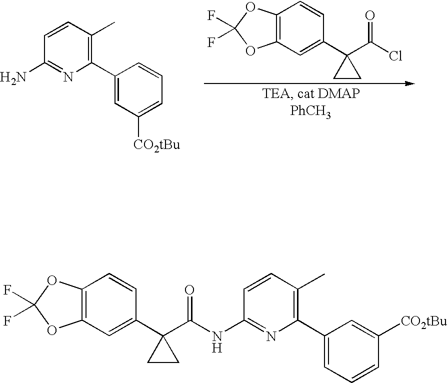

The crude acid chloride is dissolved in toluene (2.5 vol based on acid chloride) and added via addition funnel to a mixture of tert-butyl-3-(6-amino-3-methylpyridin-2-yl)benzoate (1 eq), dimethylaminopyridine (DMAP, 0.02 eq), and triethylamine (3.0 eq) in toluene (4 vol based on tert-butyl-3-(6-amino-3-methylpyridin-2-yl)benzoate). After 2 hours, water (4 vol based on tert-butyl-3-(6-amino-3-methylpyridin-2-yl)benzoate) is added to the reaction mixture. After stirring for 30 minutes, the layers are separated. The organic phase is then filtered and concentrated to afford a thick oil of 3-(6-(1-(2,2-difluorobenzo[d][1,3]dioxol-5-yl)cyclopropanecarboxamido)-3-methylpyridin-2-yl)-t-butylbenzoate (quantitative crude yield). MeCN (3 vol based on crude product) is added and distilled until crystallization occurs. Water (2 vol based on crude product) is added and the mixture stirred for 2 h. The solid is collected by filtration, washed with 1:1 (by volume) MeCN/water (2×1 vol based on crude product), and partially dried on the filter under vacuum. The solid is dried to constant weight (<1% difference) in a vacuum oven at 60° C. with a slight N₂ bleed to afford 3-(6-(1-(2,2-difluorobenzo[d][1,3]dioxol-5-yl)cyclopropanecarboxamido)-3-methylpyridin-2-yl)-t-butylbenzoate as a brown solid.

### Syntheisis of 3-(6-(1-(2,2-difluorobenzo[d][1,3]dioxol-5-yl)cyclopropanecarboxamido)-3-methylpyridin-2-yl)benzoic acid.HCL salt

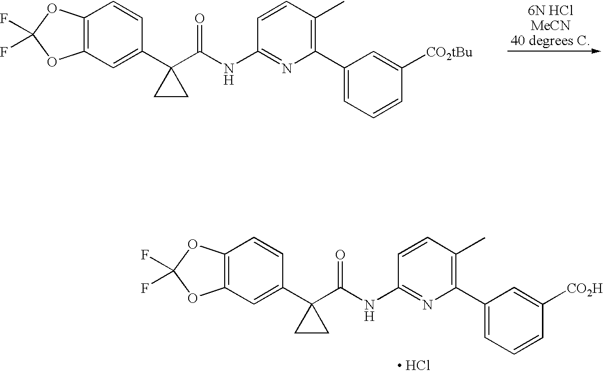

To a slurry of 3-(6-(1-(2,2-difluorobenzo[d][1,3]dioxol-5-yl)cyclopropanecarboxamido)-3-methylpyridin-2-yl)-t-butylbenzoate (1.0 eq) in MeCN (3.0 vol) is added water (0.83 vol) followed by concentrated aqueous HCl (0.83 vol). The mixture is heated to 45±5° C. After stirring for 24 to 48 hours the reaction is complete and the mixture is allowed to cool to ambient. Water (1.33 vol) is added and the mixture stirred. The solid is collected by filtration, washed with water (2×0.3 vol), and partially dried on the filter under vacuum. The solid is dried to constant weight (<1% difference) in a vacuum oven at 60° C. with a slight N₂ bleed to afford 3-(6-(1-(2,2-difluorobenzo[d][1,3]dioxol-5-yl)cyclopropanecarboxamido)-3-methylpyridin-2-yl)benzoic acid.HCl as an off-white solid.

### Synthesis of 3-(6-(1-(2,2-difluorobenzo[d][1,3]dioxol-5-yl)cyclopropanecarboxamido)-3-methylpyridin-2-yl)benzoic acid (Compound 1)

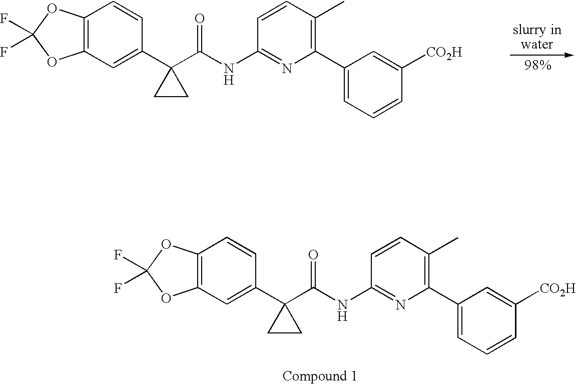

A slurry of 3-(6-(1-(2,2-difluorobenzo[d][1,3]dioxol-5-yl)cyclopropanecarboxamido)-3-methylpyridin-2-yl)benzoic acid.HCl (1 eq) in water (10 vol) is stirred at ambient temperature. A sample is taken after stirring for 24 hours. The sample is filtered and the solid washed with water (2×). The solid sample is submitted for DSC analysis. When DSC analysis indicates complete conversion to Compound 1, the solid is collected by filtration, washed with water (2×1.0 vol), and partially dried on the filter under vacuum. The solid is dried to constant weight (<1% difference) in a vacuum oven at 60° C. with a slight N₂ bleed to afford Compound 1 as an off-white solid (98% yield).

### Synthesis of 3-(6-(1-(2,2-difluorobenzo[d][1,3]dioxol-5-yl)cyclopropanecarboxamido)-3-methylpyridin-2-yl)benzoic acid (Compound 1) Using Water and Base

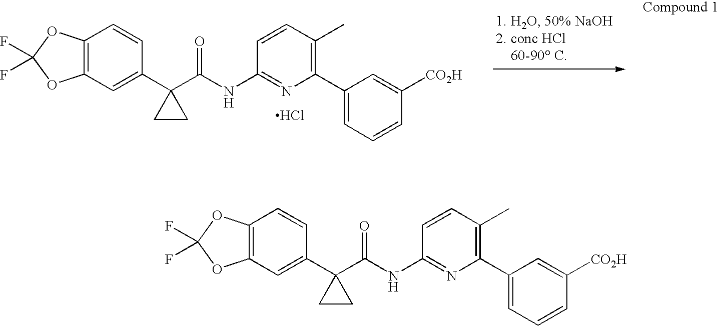

To a slurry of 3-(6-(1-(2,2-difluorobenzo[d][1,3]dioxol-5-yl)cyclopropanecarboxamido)-3-methylpyridin-2-yl)benzoic acid.HCl (1 eq) in water (10 vol) stirred at ambient temperature is added 50% w/w aq. NaOH (2.5 eq). The mixture is stirred for NLT 15 min or until a homogeneous solution. Concentrated HCl (4 eq) is added to crystallize Compound 1. The mixture is heated to 60° C. or 90° C. if needed to reduce the level of the t-butylbenzoate ester. The mixture is heated until HPLC analysis indicates NMT 0.8% (AUC) t-butylbenzoate ester. The mixture is then cooled to ambient and the solid is collected by filtration, washed with water (3×3.4 vol), and partially dried on the filter under vacuum. The solid is dried to constant weight (<1% difference) in a vacuum oven at 60° C. with a slight N₂ bleed to afford Compound 1 as an off-white solid (97% yield).

### Synthesis of 3-(6-(1-(2,2-difluorobenzo[d][1,3]dioxol-5-yl)cyclopropanecarboxamido)-3-methylpyridin-2-yl)benzoic acid (Form I) Directly from benzoate

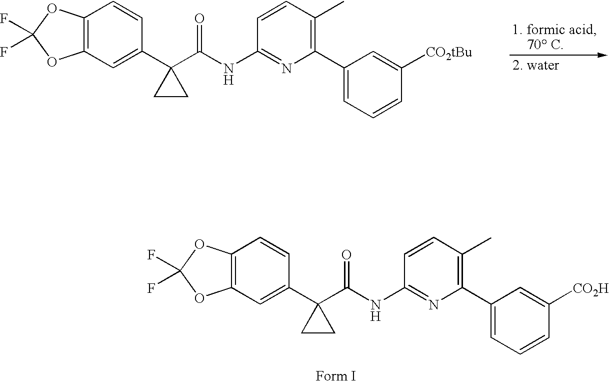

A solution of 3-(6-(1-(2,2-difluorobenzo[d][1,3]dioxol-5-yl)cyclopropanecarboxamido)-3-methylpyridin-2-yl)-t-butylbenzoate (1.0 eq) in formic acid (3.0 vol) is heated to 70±10° C. The reaction is continued until the reaction is complete (NMT 1.0% AUC 3-(6-(1-(2,2-difluorobenzo[d][1,3]dioxol-5-yl)cyclopropanecarboxamido)-3-methylpyridin-2-yl)-t-butylbenzoate) or heating for NMT 8 h. The mixture is allowed to cool to ambient. The solution is added to water (6 vol) heated at 50° C. and the mixture stirred. The mixture is then heated to 70±10° C. until the level of 3-(6-(1-(2,2-difluorobenzo[d][1,3]dioxol-5-yl)cyclopropanecarboxamido)-3-methylpyridin-2-yl)-t-butylbenzoate is NMT 0.8% (AUC). The solid is collected by filtration, washed with water (2×3 vol), and partially dried on the filter under vacuum. The solid is dried to constant weight (<1% difference) in a vacuum oven at 60° C. with a slight N₂ bleed to afford Compound 1 in Form I as an off-white solid.

An X-ray diffraction pattern calculated from a single crystal structure of Compound 1 in Form I is shown in FIG. 1. Table 10 lists the calculated peaks for FIG. 1.

TABLE 10

| Peak | 2θ Angle | Relative Intensity |
|---|---|---|
| Rank | [degrees] | [%] |
| 11 | 14.41 | 48.2 |
| 8 | 14.64 | 58.8 |
| 1 | 15.23 | 100.0 |
| 2 | 16.11 | 94.7 |
| 3 | 17.67 | 81.9 |
| 7 | 19.32 | 61.3 |
| 4 | 21.67 | 76.5 |
| 5 | 23.40 | 68.7 |
| 9 | 23.99 | 50.8 |
| 6 | 26.10 | 67.4 |
| 10 | 28.54 | 50.1 |

An actual X-ray powder diffraction pattern of Compound 1 in Form I is shown in FIG. 2. Table 11 lists the actual peaks for FIG. 2.

TABLE 11

| Peak | 2θ Angle | Relative Intensity |
|---|---|---|
| Rank | [degrees] | [%] |
| 7 | 7.83 | 37.7 |
| 3 | 14.51 | 74.9 |
| 4 | 14.78 | 73.5 |
| 1 | 15.39 | 100.0 |
| 2 | 16.26 | 75.6 |
| 6 | 16.62 | 42.6 |
| 5 | 17.81 | 70.9 |
| 9 | 21.59 | 36.6 |
| 10 | 23.32 | 34.8 |
| 11 | 24.93 | 26.4 |
| 8 | 25.99 | 36.9 |

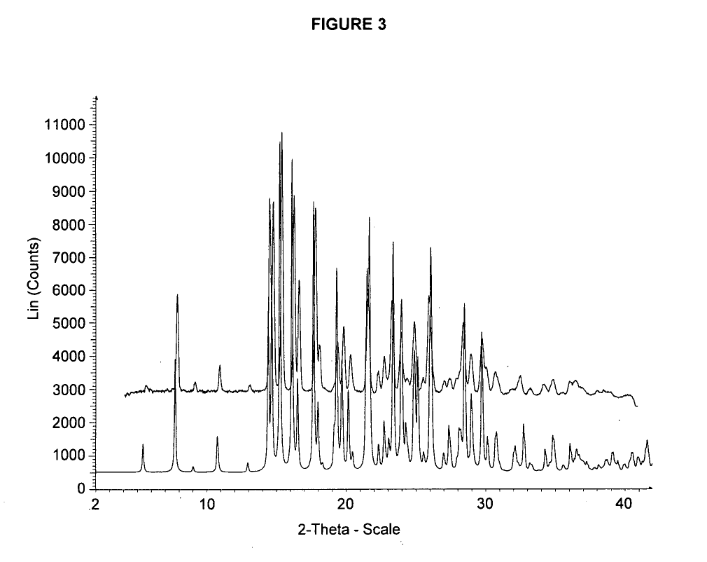

An overlay of an X-ray diffraction pattern calculated from a single crystal structure of Compound 1 in Form I, and an actual X-ray powder diffraction pattern of Compound 1 in Form Iis shown in FIG. 3. The overlay shows good agreement between the calculated and actual peak positions, the difference being only about 0.15 degrees.

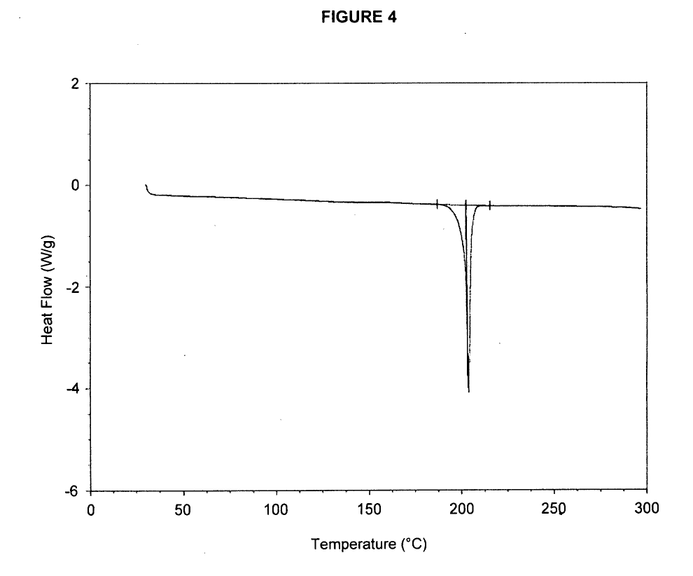

The DSC trace of Compound 1 in Form I is shown in FIG. 4. Melting for Compound 1 in Form I occurs at about 204° C.

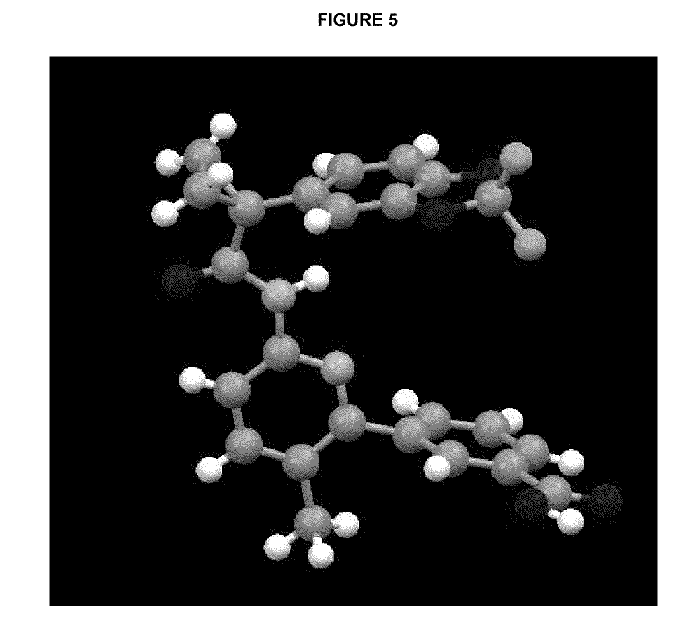

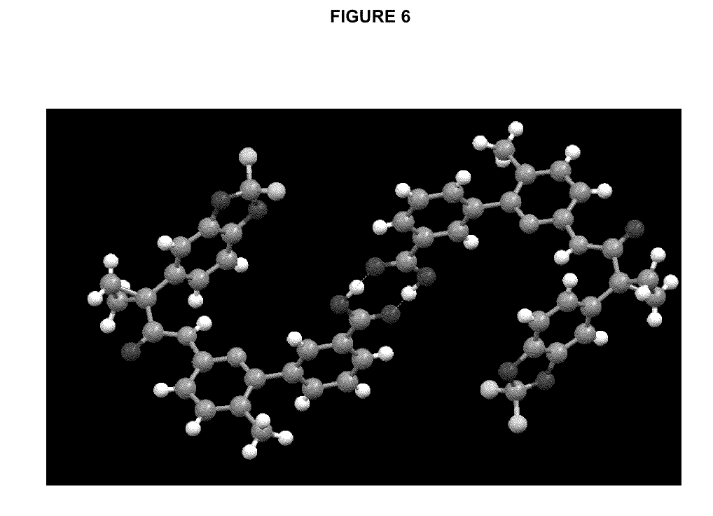

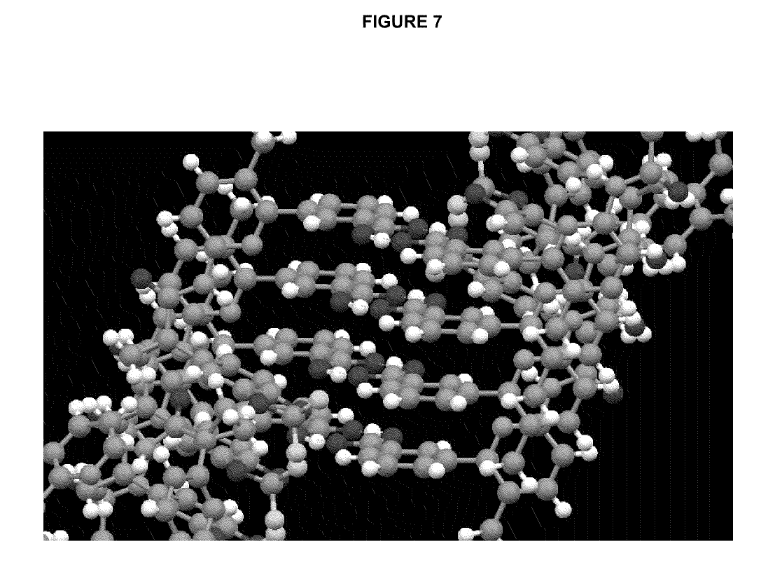

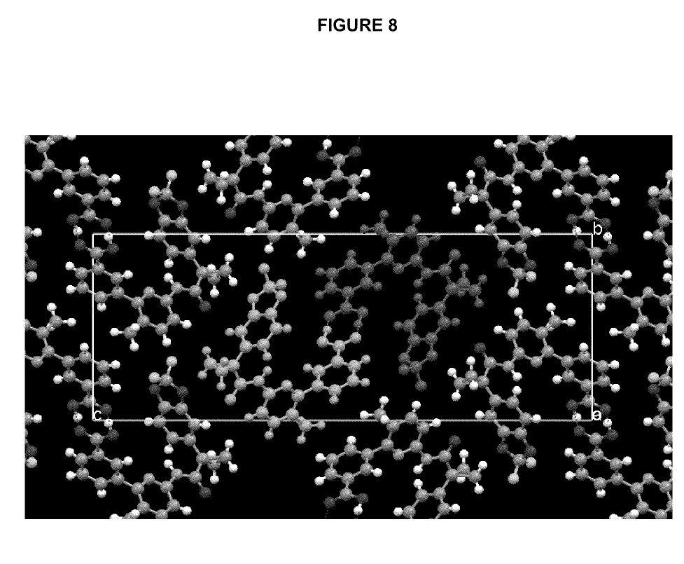

Conformational pictures of Compound 1 in Form I based on single crystal X-ray analysis are shown in FIGS. 5-8. FIGS. 6-8 show hydrogen bonding between carboxylic acid groups of a dimer and the resulting stacking that occurs in the crystal. The crystal structure reveals a dense packing of the molecules. Compound 1 in Form I is monoclinic, P2₁/n, with the following unit cell dimensions: a=4.9626(7) Å, b=12.299(2) Å, c=33.075 (4) Å, □=93.938(9)°, V=2014.0 Å³, Z=4. Density of Compound 1 in Form I calculated from structural data is 1.492 g/cm³ at 100 K.

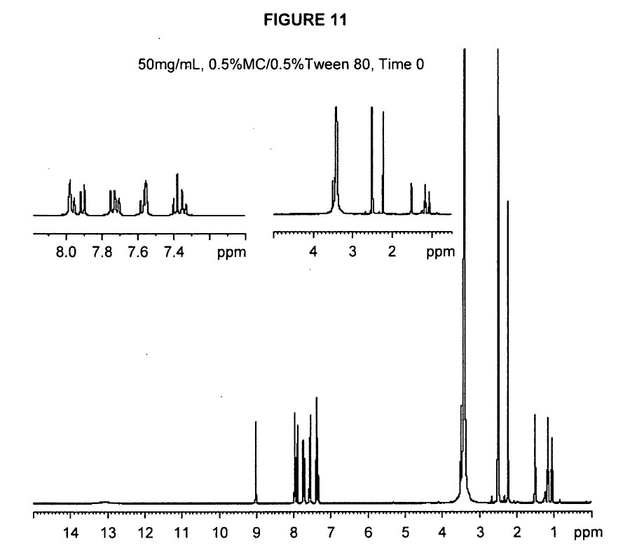

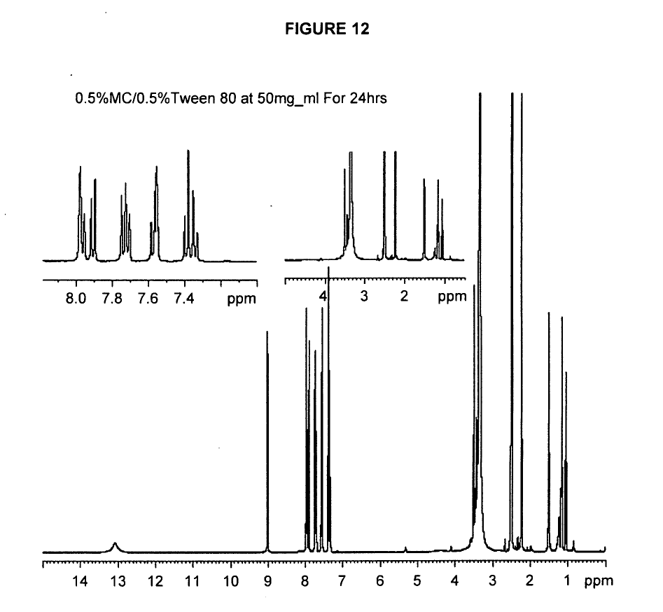

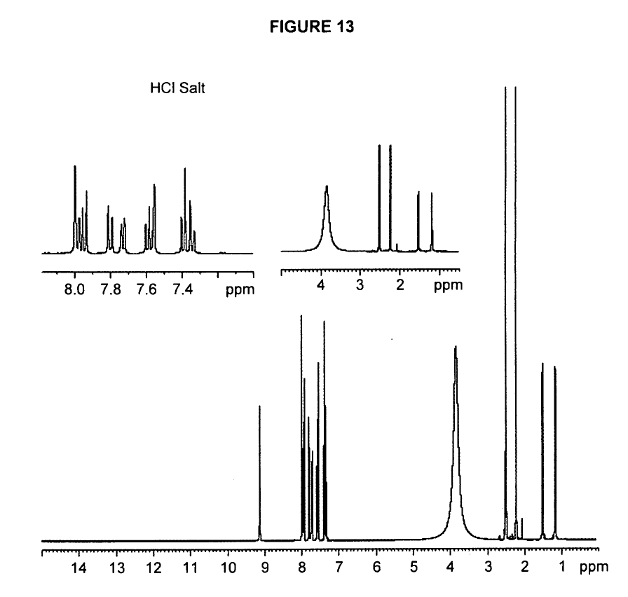

¹HNMR spectra of Compound 1 are shown in FIGS. 11-13 (FIGS. 11 and 12 depict Compound 1 in Form I in a 50 mg/mL, 0.5 methyl cellulose-polysorbate 80 suspension, and FIG. 13 depicts Compound 1 as an HCl salt).

Table 12 below recites the analytical data for Compound 1.

TABLE 12

| Cmpd. | LC/MS | LC/RT | |
|---|---|---|---|
| No. | M + 1 | min | NMR |
| 1 | 453.3 | 1.93 | H NMR (400 MHz, DMSO-d6) 9.14 (s, |
| | | | 1H), 7.99-7.93 (m, 3H), 7.80-7.78 (m, |
| | | | 1H), 7.74-7.72 (m, 1H), 7.60-7.55 (m, |
| | | | 2H), 7.41-7.33 (m, 2H), 2.24 (s, 3H), |
| | | | 1.53-1.51 (m, 2H), 1.19-1.17 (m, 2H) |

Assays for testing the salt form of 3-(6-(1-(2,2-difluorobenzo[d][1,3]dioxol-5-yl)cyclopropanecarboxamido)-3-methylpyridin-2-yl)benzoic acid and Compound 1 as CFTR modulators are disclosed in International PCT Publication WO 2007056341 (said publication being incorporated herein by reference in its entirety).

Preparation of Aqueous Formulation of Compound 1 in Form I

Because of the greater thermodynamic stability of Compound 1 over 3-(6-(1-(2,2-difluorobenzo[d][1,3]dioxol-5-yl)cyclopropanecarboxamido)-3-methylpyridin-2-yl)benzoic acid.HCl, aqueous formulations of Compound 1 can be prepared by dispersing either compound in an aqueous formulation.

### From 3-(6-(1-(2,2-difluorobenzo[d][1,3]dioxol-5-yl)cyclopropanecarboxamido)-3-methylpyridin-2-yl)benzoic acid.HCl

1. Aqueous Formulation with Methylcellulose

A 100 mL stock solution of 0.5% by weight methylcellulose was prepared by stirring 0.5 g of methylcellulose with 99.5 g of purified water until completely dissolved (approximately 24 hours). The appropriate amount of 3-(6-(1-(2,2-difluorobenzo[d][1,3]dioxol-5-yl)cyclopropanecarboxamido)-3-methylpyridin-2-yl)benzoic acid.HCl based on free base was weighed and transferred to a scintillation vial. The desired amount of 0.5% methylcellulose stock solution for making a 6 mg/mL based on free base (6.48 mg/mL based on HCl salt) was transferred into the vial and sonicated for 20 minutes and homogenized for approximately 5 minutes.

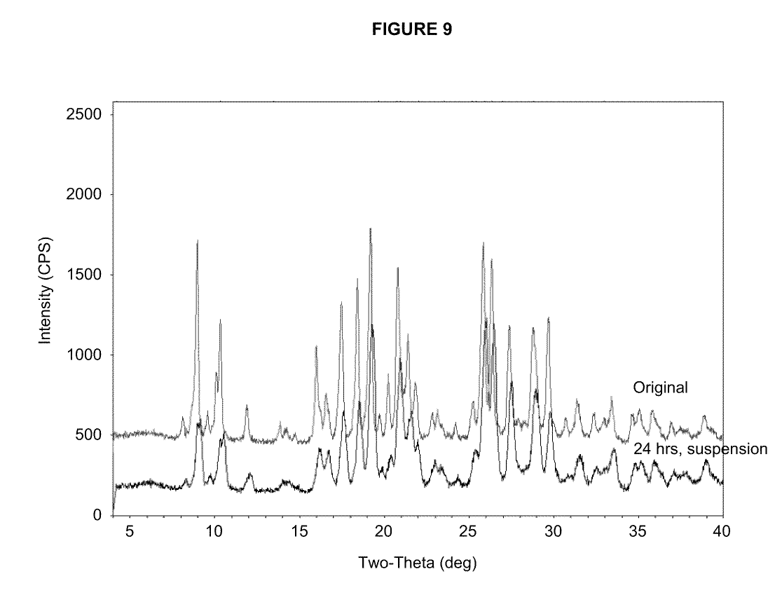

The XRPD data (FIG. 9) showed that the original solid and suspension formulation X-ray patterns are similar indicating no obvious physical change in the crystalline structure of the compound at room temperature for at least 24 hours, although formation of Compound 1 was apparent. The methylcellulose formulation was also subjected to HPLC analysis at 0 and 24 hours:

| Column: | Waters Symmetry C18, 3.5 μm, 150 * 4.60 mm, | | |
|---|---|---|---|
| | P/No: WAT200632 | | |
| Column Temperature: | not controlled | | |
| Injection Volume: | 5 μL | | |
| Flow rate: | 1 mL/min | | |
| Mobile phase: | A - 0.1% Formic Acid in water | | |
| | B - 0.1% Formic Acid in CAN | | |
| | Time | % A | % B |
| Gradient: | 0 | 75 | 25 |
| | 20′ | 10 | 90 |
| | 25′ | 10 | 90 |

| | |
|---|---|
| Post time: | 5′ |
| Detection | UV 240 nm, BW: 16 nm, Reference = 360, 100 |

Table 13. Chemical purity of 6 mg/mL aqueous methylcellulose suspension of Compound 1 as a function of storage time at room temperature.

| Time (h) | Peak purity % |
|---|---|
| T(0), sample | 99.41 |
| T(24 h), sample | 99.37 |

Compound 1 is physically and chemically stable for at least 24 hrs at room temperature in a methylcellulose formulation with no sign of chemical degradation.

2. Aqueous Formulation with Methylcellulose and Polysorbate 80

Methylcellulose (0.5 g) was combined with 99.0 g of purified water in a beaker and stirred in a 60-70° C. water bath for 30′-1 hr. The solution was stirred in a 0° C. ice/water bath for another 30′ or until clear. Polysorbate 80 (0.5 g) was added and stirring at room temperature followed for 30′-1 hr or until a clear solution was obtained.

The appropriate amount of 3-(6-(1-(2,2-difluorobenzo[d][1,3]dioxol-5-yl)cyclopropanecarboxamido)-3-methylpyridin-2-yl)benzoic acid.HCl based on free base was weighed and transferred to a scintillation vial. The desired amount of 0.5% methylcellulose and 0.5% polysorbate 80 stock solution for making a 6 mg/mL based on free base (6.48 mg/mL based on HCl salt) was transferred into the vial and sonicated for 20 minutes with alternate stirring for 1-2 minutes. The solution was homogenized for approximately 1-2 minutes.

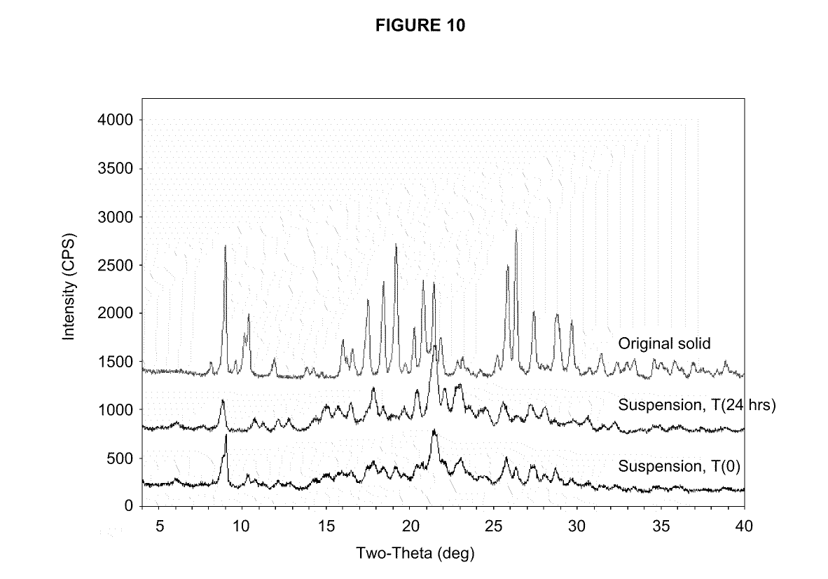

As with 0.5% methylcellulose formulation prepared previously, the HCl salt was quickly converted to Compound 1 in Form I at T(0) resulting in a crystalline free form suspension as shown by XRPD (FIG. 10) and confirmed by ¹H NMR analysis (FIGS. 11-13). Additionally, the solid form in suspension at T(0) was recovered an subjected to HPLC analysis:

| Column: | Waters Symmetry C18, 3.5 μm, 150 * 4.60 mm, | | |
|---|---|---|---|
| | P/No: WAT200632 | | |
| Column Temperature: | not controlled | | |
| Injection Volume: | 10 μL | | |
| Flow rate: | 1 mL/min | | |
| Mobile phase: | A - 0.1% Formic Acid in water | | |
| | B - 0.1% Formic Acid in CAN | | |
| | Time | % A | % B |
| Gradient: | 0 | 75 | 25 |
| | 20′ | 10 | 90 |
| | 25′ | 10 | 90 |

| | |
|---|---|
| Post time: | 5′ |
| Detection | UV 215 nm, Reference = off |

No major degradation peaks were detected and the HPLC retention time for the sample was the same as the standard used, suggesting that the differences in the XRPD pattern and ¹H NMR data between the original solid and the suspension form were not due to formation of degradents.

TABLE 14
Chemical purity of 6 mg/mL aqueous methylcellulose-
polysorbate 80 suspension of Compound 1 as a function
of storage time at room temperature.

| Time (h) | Peak purity % |
|---|---|
| T(0), sample | 98.81 |
| T(24 h), sample | 98.24 |

The Compound 1 suspension in 0.5% methylcellulose/0.5% polysorbate 80 was also tested for particle size distribution using a Malvern Master-Sizer. The suspension sample was kept at room temperature for 24 hours. As shown in Table 15, the average size of the suspension particles after 24 hours was below 10 microns.

TABLE 15
Particle size distribution of the Compound 1 suspension.

| | Particle Size (μm) | | |
|---|---|---|---|
| Time (hrs) | d10 | d50 | d90 |
| T(24 hr), sample | 2.271 | 9.792 | 49.130 |

3-(6-(1-(2,2-difluorobenzo[d][1,3]dioxol-5-yl)cyclopropanecarboxamido)-3-methylpyridin-2-yl)benzoic acid.HCl suspension in 0.5% methylcellulose/0.5% polysorbate 80 is not physically stable. The HCl salt form was quickly converted to Compound 1 in the suspension vehicle at T(0) resulting in a crystalline free form suspension. Compound 1 is chemically stable for at least 24 hrs at room temperature in 0.5% methylcellulose/0.5% polysorbate 80 formulation vehicle with no sign of chemical degradation.

Preparation of Aqueous Formulation of Compound 1 in Form I for Toxicology Studies in Animals

Starting Materials

Oral formulations of the present invention for animal toxicology testing were prepared in a standardized way using the following starting materials:

| Matrerial | Description | Manufacturer | Comments |
|---|---|---|---|
| Compound 1 | White powder | | Store at room |
| (Form I) | | | temperature |
| MW = 452 Da | | | in closed |
| | | | container |
| Methylcellulose | White powder | Sigma Aldrich | Store at room |
| 400 cP (MC) | | M0430-500G | temperature |
| | | | in closed |
| | | | container |
| Polyoxyethylene | Yellow viscous | Sigma | Store at room |
| sorbitan monoelate | liquid | P1754 | temperature |
| (Polysorbate 80) | | | in closed |
| | | | container |

Stock Vehicle Solution

The stock aqueous vehicle of methylcellulose (0.5% by weight) and polysorbate 80 (0.5% by weight) were prepared according to the following steps.

1. Add 0.5 g of methylcellulose in 33.0 g of water that has been heated to 70-80° C. and stir until the polymer is completely dispersed.

2. Remove vehicle from heat, and then add 66.0 g of water cooled to 2-8° C. while stirring. Continue stirring for 1 hour.

3. Add 0.5 g of polysorbate 80 to the above solution.

4. Stir the mixture at room temperature until the polysorbate 80 is completely dissolved (approximately 1 to 2 hours).

Amount of Compound 1 Used

The amount of Compound 1 used was caluculated as follows:

Amount of Compound 1 required=Target volume of solution (mL)×target concentration (mg/mL).

Volume of stock vehicle required (mL)=Target volume of solution (mL)−(amount of Compound 1 required (mg)/1000 mg/mL).

Note: the density of the formulation and vehicle is 1000 mg/mL.

Sample calculation for different doses is illustrated below:

### Example 1

Amount of Compound 1 (mg) required to prepare 35 mL of 25 mg/mL suspension (as free form)=35 (mL)×25 mg/mL=875 mg.

Volume of stock vehicle required=35 (mL)−(875/1000)=34.1 mL.

### Example 2

Amount of Compound 1 (mg) required to prepare 35 mL of 50 mg/mL suspension (as free form)=35 (mL)×50 mg/mL=1750 mg.

Volume of stock vehicle required=35 (mL)−(1750/1000)=33.25 mL.

### Example 3

Amount of Compound 1 (mg) required to prepare 35 mL of 100 mg/mL suspension (as free form)=35 (mL)×100 mg/mL=3500 mg.

Volume of stock vehicle required=35 (mL)−(3500/1000)=31.5 mL.

Preparation of Oral Formulations of Compound 1

Oral suspension formulations of Compound 1 were prepared according to the following steps:

1. Weigh required amount of Compound 1 according to the calculations described above.

2. Transfer Compound 1 into container if not weighed directly into container. Take care not to get the compound on the walls of the container.

3. Using a positive displacement pipette or syringe, add desired volume of 0.5% (w/w) MC/0.5% (w/w) polysorbate 80 vehicle into container.

4. Sonicate with occasional stirring for 5 minutes to evenly distribute the compound in the formulation vehicle.

5. Homogenize the formulation with medium to high speed for 2 to 3 minutes or till a homogenous suspension is formed.

6. Vortex and/or sonicate the formulation vehicle for 5 to 10 minutes. Care must be taken not to overheat the container containing the compound by repeating sonication, as longer sonication times will increase the temperature of the water bath.

7. To avoid foaming keep the container cool by placing it on ice.

8. Store formulations in closed containers at room temperature (25±3° C.) with constant stirring. Dosing should be completed within 24 hours of preparation, and remaining formulations should be discarded 24 hours after preparation.

9. Before dosing, homogenize the formulation with medium to high speed for 2 to 3 minutes or till a homogenous suspension is formed.

10. Stir the formulation constantly while dosing.

11. Repeat steps 9 through 10 before dosing if not dosed immediately after preparation.

Tables 16 through 23 list the dose calculations for Compound 1 used in the animal toxicology experiments prepared according to the above procedures.

TABLE 16
Dose calculations for Compound 1 formulation (25% overage) for a
14 day GLP toxicity study in rats.

| | | | | | Stock | | Suspension |
|---|---|---|---|---|---|---|---|
| | | | | Number | Vehicle | Amount of | Total |
| Dose | Dose Vol. | Conc. | Number rats | Dose Per | Needed | Drug | Volume |
| (mg/kg) | (mL/kg) | (mg/mL) | (0.35 kg/rat) | Day | (mL) | (grams) | (mL) |
| 150 | 5 | 30 | 32 | 1 | 67.9 | 2.1 | 70 |
| 300 | 5 | 60 | 32 | 1 | 65.8 | 4.2 | 70 |
| 600 | 5 | 120 | 32 | 1 | 61.6 | 8.4 | 70 |

TABLE 17
Dose calculations for Compound 1 formulation (25% overage) for a
14 day GLP toxicity study in dogs.

| | | | | | Stock | | Suspension |
|---|---|---|---|---|---|---|---|
| | | | | Number | Vehicle | Amount of | Total |
| Dose | Dose Vol. | Conc. | Number rats | Dose Per | Needed | Drug | Volume |
| (mg/kg) | (mL/kg) | (mg/mL) | (0.35 kg/rat) | Day | (mL) | (grams) | (mL) |
| 50 | 5 | 10 | 6 | 1 | 371.25 | 3.75 | 375 |
| 100 | 5 | 20 | 6 | 1 | 367.50 | 7.50 | 375 |
| 200 | 5 | 40 | 6 | 1 | 360.00 | 15.0 | 375 |

TABLE 18
Dose calculations for Compound 1 formulation (25% overage) for an
acute GLP toxicity study in rats.

| | | | | | Stock | | Suspension |
|---|---|---|---|---|---|---|---|
| | | | | Number | Vehicle | Amount of | Total |
| Dose | Dose Vol. | Conc. | Number rats | Dose Per | Needed | Drug | Volume |
| (mg/kg) | (mL/kg) | (mg/mL) | (0.35 kg/rat) | Day | (mL) | (grams) | (mL) |
| 500 | 10 | 50 | 32 | 1 | 133 | 7.0 | 140 |
| 1000 | 10 | 100 | 32 | 1 | 126 | 14.0 | 140 |
| 2000 | 10 | 200 | 32 | 1 | 112 | 28.0 | 140 |

TABLE 19
Dose calculations for Compound 1 formulation (25% overage) for an
acute GLP toxicit study in mice.

| | | | | | Stock | | Suspension |
|---|---|---|---|---|---|---|---|
| | | | | Number | Vehicle | Amount of | Total |
| Dose | Dose Vol. | Conc. | Number rats | Dose Per | Needed | Drug | Volume |
| (mg/kg) | (mL/kg) | (mg/mL) | (0.35 kg/rat) | Day | (mL) | (grams) | (mL) |
| 500 | 20 | 25 | 76 | 1 | 58.5 | 1.5 | 60 |
| 1000 | 20 | 50 | 76 | 1 | 57.0 | 3.0 | 60 |
| 2000 | 20 | 100 | 76 | 1 | 54.0 | 6.0 | 60 |

TABLE 20
Dose calculations for Compound 1 formulation (25% overage) for an
oral telemetry study in dogs.

| | | | | | Stock | | Suspension |
|---|---|---|---|---|---|---|---|
| | | | | Number | Vehicle | Amount of | Total |
| Dose | Dose Vol. | Conc. | Number rats | Dose Per | Needed | Drug | Volume |
| (mg/kg) | (mL/kg) | (mg/mL) | (0.35 kg/rat) | Day | (mL) | (grams) | (mL) |
| 50 | 5 | 10 | 4 | 1 | 247.5 | 2.5 | 250 |
| 100 | 5 | 20 | 4 | 1 | 245.0 | 5.0 | 250 |
| 200 | 5 | 40 | 4 | 1 | 240.0 | 10.0 | 250 |

TABLE 21
Dose calculations for Compound 1 formulation (25% overage) for an
in vivo GLP genotoxicity study in mice.

| | | | | | Stock | | Suspension |
|---|---|---|---|---|---|---|---|
| | | | | Number | Vehicle | Amount of | Total |
| Dose | Dose Vol. | Conc. | Number rats | Dose Per | Needed | Drug | Volume |
| (mg/kg) | (mL/kg) | (mg/mL) | (0.35 kg/rat) | Day | (mL) | (grams) | (mL) |
| 500 | 20 | 25 | 10 | 1 | 7.8 | 0.20 | 8.0 |
| 1000 | 20 | 50 | 10 | 1 | 7.6 | 0.40 | 8.0 |
| 2000 | 20 | 100 | 10 | 1 | 7.2 | 0.80 | 8.0 |

TABLE 22
Dose calculations for Compound 1 formulation (25% overage) for a
GLP toxicity study in rats (Irwin).

| | | | | | Stock | | Suspension |
|---|---|---|---|---|---|---|---|
| | | | | Number | Vehicle | Amount of | Total |
| Dose | Dose Vol. | Conc. | Number rats | Dose Per | Needed | Drug | Volume |
| (mg/kg) | (mL/kg) | (mg/mL) | (0.35 kg/rat) | Day | (mL) | (grams) | (mL) |
| 250 | 10 | 25 | 8 | 1 | 34.10 | 0.875 | 35 |
| 500 | 10 | 50 | 8 | 1 | 33.25 | 1.75 | 35 |
| 1000 | 10 | 100 | 8 | 1 | 31.5 | 3.50 | 35 |

TABLE 23
Dose calculations for Compound 1 formulation (25% overage) for a
GLP toxicity study in rats (respiratory & GI motility).

| | | | | | Stock | | Suspension |
|---|---|---|---|---|---|---|---|
| | | | | Number | Vehicle | Amount of | Total |
| Dose | Dose Vol. | Conc. | Number rats | Dose Per | Needed | Drug | Volume |
| (mg/kg) | (mL/kg) | (mg/mL) | (0.35 kg/rat) | Day | (mL) | (grams) | (mL) |
| 250 | 10 | 25 | 10 | 1 | 42.9 | 1.10 | 44 |
| 500 | 10 | 50 | 10 | 1 | 41.8 | 2.20 | 44 |
| 1000 | 10 | 100 | 10 | 1 | 39.6 | 4.40 | 44 |

We claim:

1. An aqueous formulation comprising 3-(6-(1-(2,2-difluorobenzo[d][1,3]dioxol-5-yl)cyclopropanecarboxamido)-3-methylpyridin-2-yl)benzoic acid, water, and a viscosity agent.

2. The formulation of claim 1, wherein the 3-(6-(1-(2,2-difluorobenzo[d][1,3]dioxol-5-yl)cyclopropanecarboxamido)-3-methylpyridin-2-yl)benzoic acid is characterized by one or more peaks at 15.2 to 15.6 degrees, 16.1 to 16.5 degrees, and 14.3 to 14.7 degrees in an X-ray powder diffraction obtained using Cu K alpha radiation.

3. The formulation of claim 2, wherein the 3-(6-(1-(2,2-difluorobenzo[d][1,3]dioxol-5-yl)cyclopropanecarboxamido)-3-methylpyridin-2-yl)benzoic acid is characterized by one or more peaks at 15.4, 16.3, and 14.5 degrees.

4. The formulation of claim 2, wherein the 3-(6-(1-(2,2-difluorobenzo[d][1,3]dioxol-5-yl)cyclopropanecarboxamido)-3-methylpyridin-2-yl)benzoic acid is further characterized by a peak at 14.6 to 15.0 degrees.

5. The formulation of claim 4, wherein the 3-(6-(1-(2,2-difluorobenzo[d][1,3]dioxol-5-yl)cyclopropanecarboxamido)-3-methylpyridin-2-yl)benzoic acid is further characterized by a peak at 14.8 degrees.

6. The formulation of claim 4, wherein the 3-(6-(1-(2,2-difluorobenzo[d][1,3]dioxol-5-yl)cyclopropanecarboxamido)-3-methylpyridin-2-yl)benzoic acid is further characterized by a peak at 17.6 to 18.0 degrees.

7. The formulation of claim 6, wherein the 3-(6-(1-(2,2-difluorobenzo[d][1,3]dioxol-5-yl)cyclopropanecarboxamido)-3-methylpyridin-2-yl)benzoic acid is further characterized by a peak at 17.8 degrees.

8. The formulation of claim 6, wherein the 3-(6-(1-(2,2-difluorobenzo[d][1,3]dioxol-5-yl)cyclopropanecarboxamido)-3-methylpyridin-2-yl)benzoic acid is further characterized by a peak at 16.4 to 16.8 degrees.

9. The formulation of claim 8, wherein the 3-(6-(1-(2,2-difluorobenzo[d][1,3]dioxol-5-yl)cyclopropanecarboxamido)-3-methylpyridin-2-yl)benzoic acid is further characterized by a peak at 16.4 to 16.8 degrees.

10. The formulation of claim 9, wherein the 3-(6-(1-(2,2-difluorobenzo[d][1,3]dioxol-5-yl)cyclopropanecarboxamido)-3-methylpyridin-2-yl)benzoic acid is further characterized by a peak at 16.6 degrees.

11. The formulation of claim 9, wherein the 3-(6-(1-(2,2-difluorobenzo[d][1,3]dioxol-5-yl)cyclopropanecarboxamido)-3-methylpyridin-2-yl)benzoic acid is further characterized by a peak at 7.6 to 8.0 degrees.

12. The formulation of claim 11, wherein the 3-(6-(1-(2,2-difluorobenzo[d][1,3]dioxol-5-yl)cyclopropanecarboxamido)-3-methylpyridin-2-yl)benzoic acid is further characterized by a peak at 7.8 degrees.

13. The formulation of claim 11, wherein the 3-(6-(1-(2,2-difluorobenzo[d][1,3]dioxol-5-yl)cyclopropanecarboxamido)-3-methylpyridin-2-yl)benzoic acid is further characterized by a peak at 25.8 to 26.2 degrees.

14. The formulation of claim 13, wherein the 3-(6-(1-(2,2-difluorobenzo[d][1,3]dioxol-5-yl)cyclopropanecarboxamido)-3-methylpyridin-2-yl)benzoic acid is further characterized by a peak at 26.0 degrees.

15. The formulation of claim 13, wherein the 3-(6-(1-(2,2-difluorobenzo[d][1,3]dioxol-5-yl)cyclopropanecarboxamido)-3-methylpyridin-2-yl)benzoic acid is further characterized by a peak at 21.4 to 21.8 degrees.

16. The formulation of claim 15, wherein the 3-(6-(1-(2,2-difluorobenzo[d][1,3]dioxol-5-yl)cyclopropanecarboxamido)-3-methylpyridin-2-yl)benzoic acid is further characterized by a peak at 21.6 degrees.

17. The formulation of claim 15, wherein the 3-(6-(1-(2,2-difluorobenzo[d][1,3]dioxol-5-yl)cyclopropanecarboxamido)-3-methylpyridin-2-yl)benzoic acid is further characterized by a peak at 23.1 to 23.5 degrees.

18. The formulation of claim 17, wherein the 3-(6-(1-(2,2-difluorobenzo[d][1,3]dioxol-5-yl)cyclopropanecarboxamido)-3-methylpyridin-2-yl)benzoic acid is further characterized by a peak at 23.3 degrees.

19. The formulation of claim 1, wherein the 3-(6-(1-(2,2-difluorobenzo[d][1,3]dioxol-5-yl)cyclopropanecarboxamido)-3-methylpyridin-2-yl)benzoic acid is characterized by a diffraction pattern substantially similar to that of FIG. 1.

20. The formulation of claim 1, wherein the 3-(6-(1-(2,2-difluorobenzo[d][1,3]dioxol-5-yl)cyclopropanecarboxamido)-3-methylpyridin-2-yl)benzoic acid is characterized by a diffraction pattern substantially similar to that of FIG. 2.

21. The formulation of claim 1, wherein the 3-(6-(1-(2,2-difluorobenzo[d][1,3]dioxol-5-yl)cyclopropanecarboxamido)-3-methylpyridin-2-yl)benzoic acid has a monoclinic crystal system, a P2₁/n space group, and the following unit cell dimensions:
a=4.9626 (7) Å α=90°
b=12.2994 (18) Å β=93.938 (9)°
c=33.075 (4) Å γ=90°.

22. The formulation of claim 1, wherein the viscosity agent is selected from the group consisting of methyl cellulose, sodium carboxymethylcellulose, hydroxypropylmethyl cellulose, hydroxypropyl cellulose, sodium alginate, polyacrylate, povidone, acacia, guar gum, xanthan gum, tragacanth, and magnesium aluminum silicate.

23. The formulation of claim 1, wherein the viscosity agent is methylcellulose.

24. The formulation of claim 1, wherein the concentration of 3-(6-(1-(2,2-difluorobenzo[d][1,3]dioxol-5-yl)cyclopropanecarboxamido)-3-methylpyridin-2-yl)benzoic acid is from 0.5 to 20% by weight.

25. The formulation of claim 1, wherein the concentration of 3-(6-(1-(2,2-difluorobenzo[d][1,3]dioxol-5-yl)cyclopropanecarboxamido)-3-methylpyridin-2-yl)benzoic acid is from 2.5 to 3.5% by weight.

26. The formulation of claim 1, wherein the concentration of viscosity agent is from 0.1 to 2% by weight.

27. The formulation of claim 1, wherein the concentration of viscosity agent is 0.5% by weight.

28. The formulation of claim 1, wherein the concentration of 3-(6-(1-(2,2-difluorobenzo[d][1,3]dioxol-5-yl)cyclopropanecarboxamido)-3-methylpyridin-2-yl)benzoic acid is from 0.5 to 20% by weight; and the concentration of viscosity agent is from 0.1 to 2% by weight.

29. The formulation of claim 1, wherein the concentration of 3-(6-(1-(2,2-difluorobenzo[d][1,3]dioxol-5-yl)cyclopropanecarboxamido)-3-methylpyridin-2-yl)benzoic acid is from 2.5 to 3.5% by weight; and the concentration of viscosity agent is 0.5% by weight.

30. The formulation of claim 1 further comprising a surfactant.

31. The formulation of claim 30, wherein the surfactant is an anionic, cationic, or nonionic surfactant.

32. The formulation of claim 31, wherein the surfactant is an anionic surfactant selected from the group consisting of salts of dodecyl sulfate, lauryl sulfate, laureth sulfate, alkyl benzene sulfonates, butanoic acid, hexanoic acid, octanoic acid, decanoic acid, lauric acid, myristic acid, palmitic acid, stearic acid, arachidic acid, behenic acid, myristoleic acid, palmitoleic acid, oleic acid, linoleic acid, alpha-linolenic acid, arachidonic acid, eicosapentaenoic acid, erucic acid, and docosahexaenoic acid.

33. The formulation of claim 31, wherein the surfactant is a cationic surfactant selected from the group consisting of cetyl trimethylammonium bromide, cetylpyridinium chloride, polethoxylated tallow amine, benzalkonium chloride, and benzethonium chloride.

34. The formulation of claim 31, wherein the surfactant is a nonionic surfactant selected from the group consisting of polysorbate 20, polysorbate 40, polysorbate 60, polysorbate 65, polysorbate 80, alkyl poly(ethylene oxide), poloxamine, alkyl polyglucosides, octyl glucoside, decyl maltoside, fatty alcohol, cetyl alcohol, oleyl alcohol, cocamide MEA, cocamide DEA, and cocamide TEA.

35. The formulation of claim 30, wherein the surfactant is polysorbate 80.

36. The formulation of claim 30, wherein the concentration of surfactant is from 0.1 to 10% by weight.

37. The formulation of claims 30, wherein the concentration of surfactant is 0.5% by weight.

38. The formulation of claim 30, wherein the surfactant is polysorbate 80 at 0.5% by weight.

39. The formulation of claim 1 further comprising an antifoaming agent.

40. The formulation of claim 39, wherein the antifoaming agent comprises polydimethylsiloxane.

41. The formulation of claim 40, wherein the antifoaming agent is simethicone.

42. The formulation of claim 39, wherein the concentration of antifoaming agent is from 0.01 to 0.2% by weight.

43. The formulation of claim 39, wherein the concentration of antifoaming agent is 0.05% by weight.

44. The formulation of claim 1 further comprising a buffer.

45. The formulation of claim 44, wherein the buffer comprises sodium, potassium or ammonium salt of acetic, boric, carbonic, phosphoric, succinic, malic, tartaric, citric, acetic, benzoic, lactic, glyceric, gluconic, glutaric or glutamic acids.

46. The formulation of claim 44, wherein the buffer comprises sodium, potassium or ammonium salt of citric acid.

47. The formulation of claim 1 further comprising a masking and/or flavoring agent.

48. An oral formulation comprising 3-(6-(1-(2,2-difluorobenzo[d][1,3]dioxol-5-yl)cyclopropanecarboxamido)-3-methylpyridin-2-yl)benzoic acid, water, methyl cellulose, polysorbate 80, and simethicone.

49. The oral formulation of claim 48, wherein 3-(6-(1-(2,2-difluorobenzo[d][1,3]dioxol-5-yl)cyclopropanecarboxamido)-3-methylpyridin-2-yl)benzoic acid is present in a concentration of 2.5% to 3.5% by weight.

50. The oral formulation of claim 49, wherein methyl cellulose is present in a concentration of 0.5% by weight.

51. The oral formulation of claim 50, wherein polysorbate 80 is present in a concentration of 0.5% by weight.

52. The oral formulation of claim 51, wherein simethicone is present in a concentration of 0.05% by weight.

53. A method of treating cystic fibrosis in a mammal comprising administering the formulation of claim 1.

54. The method of claim 53, wherein the method comprises administering an additional therapeutic agent.

55. The method of claim 54, wherein the additional therapeutic agent is selected from the group consisting of mucolytic agent, bronchodialator, an anti-biotic, an anti-infective agent, an anti-inflammatory agent, a CFTR modulator other than a compound of the present invention, and a nutritional agent.

56. A kit comprising the formulation of claim 1 and instructions for use thereof.
